(19) United States
(12) Patent Application Publication
(10) Pub. No.: US 2022/0205883 A1
(43) Pub. Date: Jun. 30, 2022

(54) DIAGNOSTIC CONSUMABLES INCORPORATING COATED MICRO-PROJECTION ARRAYS, AND METHODS THEREOF

(71) Applicant: Siemens Healthcare Diagnostics Inc., Tarrytown, NY (US)

(72) Inventors: Sape de Vries, Elkhart, IN (US); Nigel Fairbank, Ottawa (CA)

(73) Assignee: Siemens Healthcare Diagnostics Inc., Tarrytown, NY (US)

(21) Appl. No.: 17/593,307

(22) PCT Filed: Feb. 25, 2020
(86) PCT No.: PCT/US2020/019665

§ 371 Date: Sep. 15, 2021

Related U.S. Application Data
(60) Provisional application No. 62/819,973, filed on Mar. 18, 2019.
(60) Provisional application No. 62/875,167, filed on Jul. 17, 2019.

(51) Int. Cl. G01N 1/38; G01N 33/50; B01L 3/00

(52) U.S. Cl. CPC G01N 1/38; G01N 33/5002; B01L 3/50273; B01L 3/502715; B01L 2400/049; B01L 2300/0819; B01L 2300/0861; B01L 2300/123; B01L 2400/086; B01L 2200/16

(57) ABSTRACT

Diagnostic consumables for use in the analysis of fluid samples, such as whole blood, plasma or urine, are provided. The diagnostic consumables include a substrate having a sample preparation stage that includes an inlet port for receiving a fluid sample, an outlet port for dispensing a prepared fluid sample, and a channel extending from the inlet port to the outlet port. The channel includes an array of micro-projections extending into the channel to define a plurality of flow paths therebetween along at least a portion of a length of the channel between the inlet port and the outlet port. A material is disposed on the array of micro-projections for mixing with the fluid sample as the fluid sample is flowed through the channel to generate the prepared fluid sample. Methods of operating and manufacturing the diagnostic consumables are also provided.

This application claims priority to U.S. Provisional Application No. 62/819,973, filed on Mar. 18, 2019 and U.S. Provisional Application No. 62/875,167 filed on Jul. 17, 2019. The entire contents of the above-referenced patent applications are hereby expressly incorporated herein by reference.

### FIELD

This application relates generally to fluidic devices, and in particular to sample preparation stages for sample fluid preparation in fluidic devices.

### BACKGROUND

Fluidic devices are used to control and/or manipulate fluids for any of a variety of applications. A fluidic device could include channels that constrain the flow of a fluid in the device. A channel could also or instead be considered a microchannel if at least one dimension of the channel (a radius, width or height, for example) is sub-millimeter, and/or if the channel carries sub-milliliter volumes of fluid. A fluidic device that includes a microchannel, and/or other microscale components, could be considered a microfluidic device.

Fluidic devices could incorporate and/or be coupled to one or more sensors to provide sensing capabilities. For example, a sample fluid could be pumped through channels in a fluidic device to a sensing region of the fluidic device in order to be exposed to a sensor. The sensor could be incorporated into the fluidic device and/or part of a separate device to which the sensing region is exposed in order to measure one or more properties of the fluid. A fluidic device that incorporates one or more sensors or sensing regions could be used as a diagnostic device. In the context of medical diagnostic devices, fluidic devices could be used in the measurement of one or more properties of a bodily fluid. By way of example, a blood sample could be added to a fluidic device to control and/or manipulate the blood sample in order to measure the concentration of certain analytes in the blood.

In recent years, microfluidic devices have attracted attention for use in the field as diagnostic devices for point-of-care testing. A fluidic device in this field usually provides integration of multiple analytical steps into a single device. A fluidic device may perform one or more assays. For the purposes of the instant disclosure, an assay may be defined as a procedure for quantifying the amount or the functional activity of an analyte in a liquid sample. An assay may involve a variety of operations on the fluidic device, such as sample introduction, preparation, metering, sample/reagent mixing, liquid transport, and detection, etc.

Typical diagnostic assays involve manipulating small volumes of fluid with precise control, which can be challenging due to several factors, such as fluid loss in transport, capillary effects, impact of gravity, trapped air and others. Additionally, several assay processes such as mixing and incubation can also pose unique challenges in miniature fluidic devices. For disposable fluidic devices used as diagnostic consumables, these challenges are often compounded by the need for a solution that is both cost-effective and provides the level of precision needed to deliver the required assay performance. Improving the efficiency, reliability and repeatability of measurements is an important consideration in the design of diagnostic devices, and particularly in the context of single use diagnostic consumables compatible with a small form factor instrument.

### SUMMARY

According to a first aspect, the present disclosure provides a diagnostic consumable for use in the analysis of a fluid sample, the diagnostic consumable comprising: a substrate having a sample preparation stage, the sample preparation stage comprising: i) an inlet port for receiving a fluid sample; ii) an outlet port for dispensing a prepared fluid sample; and iii) a channel extending from the inlet port to the outlet port, the channel comprising an array of micro-projections extending into the channel to define a plurality of flow paths therebetween along at least a portion of a length of the channel between the inlet port and the outlet port, the array of micro-projections having disposed thereon a material for mixing with the fluid sample as the fluid sample is flowed through the channel to generate the prepared fluid sample.

According to a second aspect, the present disclosure provides a method for analysis of a fluid sample on a diagnostic consumable, the method comprising: receiving a fluid sample at an inlet port of a sample preparation stage of the diagnostic consumable; mixing a material into the fluid sample by flowing the fluid sample through a channel of the sample preparation stage of the diagnostic consumable, the channel comprising an array of micro-projections extending into the channel to define a plurality of flow paths therebetween along at least a portion of a length of the channel, the array of micro-projections having disposed thereon the material for mixing with the fluid sample as the fluid sample is flowed through the channel to generate a prepared fluid sample.

According to a third aspect, the present disclosure provides a method of making a diagnostic consumable for use in analysis of a fluid sample, the method comprising: obtaining a substrate that includes a channel having an array of micro-projections extending into the channel to define a plurality of flow paths therebetween along at least a portion of a length of the channel; applying a fluid to the array of micro-projections in the channel, the fluid comprising a material for deposition on the array of micro-projections; and drying-down the fluid onto the array of micro-projections so that the array of micro-projections has the material disposed thereon.

According to a fourth aspect, the present disclosure provides a diagnostic consumable for use in the analysis of whole blood. The diagnostic consumable includes a substrate having a haemolysis stage that includes an inlet port for receiving whole blood, an outlet port for dispensing haemolysed blood, and a haemolysis channel extending from the inlet port to the outlet port. The haemolysis channel includes an array of micro-projections extending into the haemolysis channel to define a plurality of flow paths therebetween along at least a portion of a length of the haemolysis channel between the inlet port and the outlet port. The array of micro-projections has disposed thereon a haemolytic reagent for interaction with the whole blood as the whole blood is flowed through the haemolysis channel to generate haemolysed blood.

According to a fifth aspect of the present disclosure, the present disclosure provides a method for analysis of a whole blood sample on a diagnostic consumable. The method includes receiving a whole blood sample at an inlet port of a haemolysis stage of the diagnostic consumable and haemolysing the whole blood by flowing the whole blood through a haemolysis channel of the haemolysis stage of the diagnostic consumable. The haemolysis channel includes an array of micro-projections extending into the haemolysis channel to define a plurality of flow paths therebetween along at least a portion of a length of the haemolysis channel. The array of micro-projections has disposed thereon a haemolytic reagent for interaction with the whole blood as the whole blood is flowed through the haemolysis channel to generate haemolysed blood.

According to a sixth aspect of the present disclosure, the present disclosure provides a method of making a diagnostic consumable for use in analysis of a whole blood sample. The method includes obtaining a substrate that includes a haemolysis channel having an array of micro-projections extending into the haemolysis channel to define a plurality of flow paths therebetween along at least a portion of a length of the haemolysis channel. The method further includes applying a haemolytic reagent solution to the array of micro-projections in the haemolysis channel and drying-down the haemolytic reagent solution onto the array of micro-projections so that the array of micro-projections has dried haemolytic reagent disposed thereon.

Other aspects and features of embodiments of the present disclosure will become apparent to those ordinarily skilled in the art upon review of the following description.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the present application, there is shown in the drawings illustrative embodiments of the disclosure. It should be understood, however, that the application is not limited to the precise arrangements and instrumentalities shown. In the drawings:

FIG. 1 is an isometric view of a haemolysis stage of a diagnostic consumable;

FIG. 2 is a magnified view of a portion of the haemolysis channel of the haemolysis stage of FIG. 1;

FIG. 3 is an isometric view of an injection molded micro-pillar;

FIG. 4 is an isometric view of the haemolysis stage of FIG. 1 with a transparent cover layer;

FIG. 5 is a a cross-sectional view of the haemolysis stage of FIG. 4, taken along the line illustrated in FIG. 4;

FIG. 6 is a plan view of a portion of the haemolysis channel of the haemolysis stage of FIG. 4;

FIG. 7 is a a cross-sectional view of the haemolysis channel of FIG. 6, taken along the line illustrated in FIG. 6 that extends transverse to the direction of flow along the haemolysis channel;

FIG. 8 is a cross-sectional view of another haemolysis channel, taken along a line that extends transverse to the direction of flow along the haemolysis channel;

FIG. 9 is a cross-sectional view of yet another haemolysis channel, taken along a line that extends transverse to the direction of flow along the haemolysis channel;

FIG. 10 is a cross-sectional view of still another haemolysis channel, taken along a line that extends transverse to the direction of flow along the haemolysis channel;

FIG. 11 is a plan view of a portion of another implementation of a haemolysis channel;

FIG. 12 is an isometric view of the top of an example substrate for a diagnostic consumable that includes the haemolysis stage of FIG. 4;

FIG. 13 is an isometric view of the bottom of the substrate of FIG. 12;

FIG. 14 is a plan view of the top of the substrate of FIG. 12;

FIG. 15 is a plan view of the bottom of the substrate of FIG. 12;

FIG. 16 is a plan view of the top of an example diagnostic consumable incorporating the substrate of FIG. 12;

FIG. 17 is a plan view of the bottom of the diagnostic consumable of FIG. 16;

FIG. 18 is a plan view of the haemolysis stage of the diagnostic consumable of FIGS. 16 and 17;

FIG. 19 is a flow diagram illustrating an example method for making a diagnostic consumable for use in analysis of a whole blood sample; and

FIG. 20 is a flow diagram illustrating an example method for analysis of a whole blood sample on a diagnostic consumable.

### DETAILED DESCRIPTION

In fluidic devices, reagent-sample fluid interaction can be provided by flowing a sample fluid through a channel in which a reagent has been dried-down on one or more walls of the channel. The incoming sample dissolves, re-suspends, and reacts with the reagent.

However, sample fluid flow within a microfluidic channel is generally laminar, which means that there is little or no turbulent mixing within the channel and the interaction between reagent and the sample is effectively limited by diffusion. If reagent is only dried-down on the walls of a channel in which the fluid flow is laminar, the concentration of reagent in the sample may initially be highest near the walls where the reagent was dried, and lower towards the centre of the channel's cross-section. For example, in the case of haemolysis, this means blood running through the centre of the channel could remain unhaemolysed until the haemolysing reagent slowly diffuses to it. This can be problematic if complete haemolysis is necessary or desirable for a subsequent analysis, such as an optical measurement for co-oximetry.

Reducing the cross-section of the channel to reduce the diffusion distance can potentially reduce the time required for complete haemolysis, but reducing the cross-section of the channel makes the channel highly flow resistive, which can complicate sample flow/delivery downstream of the channel.

The present disclosure relates, in part, to diagnostic consumables that include components or structures for mixing a material, such as a reagent, with a sample fluid. For example, some diagnostic consumables described herein include a sample preparation channel that includes an array of micro-projections that are coated in the material that is to be mixed with the fluid sample. The array of micro-projections define a plurality of flow paths along a length of the channel. The material-coated micro-projection array provides additional surface area onto which the material can be applied and may reduce the diffusion distance between the material and component(s) in the sample fluid with which the material is intended to mix and/or interact as the sample fluid is flowed through the sample preparation channel. The material may be anything that mixes with and/or interacts with the sample (or components thereof—such as an analyte of interest). Non-limiting examples include reagents, antibodies, surfactants, sample conditioning compounds, and the like. For example, in some embodiments, these diagnostic consumables could be configured for blood testing and/or analysis. In such embodiments, the material could include a reagent, e.g., a blood haemolysing reagent, such as a detergent, that haemolyses blood cells in a whole blood sample, or a coagulant, such as Celite™ (diatomaceous earth) or kaolin, that promote blood clotting. In some embodiments, these diagnostic consumables could be configured for detection of drugs of abuse. For example, in such embodiments the material may comprise a reagent that reacts with a drug of abuse such as barbituates, cannabinoids, cocaine metabolite, ethanol, ecstasy, methadone, methamphetamine and opiates. In some embodiments, these diagnostic consumables could be implemented in a small form factor, such as in the form of a diagnostic card or a test card, for example. In some embodiments, these diagnostic consumables are microfluidic devices.

The diagnostic consumables could include a substrate with other channels and/or other fluidic components formed therein. Cover layers could be applied to the substrate to seal top and/or bottom surfaces of the substrate. The substrate could also include and/or be coupled to a sensing region that includes one or more sensors. These sensors could measure one or more properties of a sample fluid, such as the concentration of certain analytes in a blood sample or the time required to reach a certain level of coagulation as part of a prothrombin time (PT) test, for example. To perform measurements, the diagnostic consumable could be inserted into an instrument such as a diagnostic consumable reader module. A blood sample could then be inserted into the diagnostic consumable. The diagnostic consumable reader module could then use and/or control the diagnostic consumable to perform measurements on the blood sample. The combination of the diagnostic consumable and the diagnostic consumable reader module could be considered a blood analysis system.

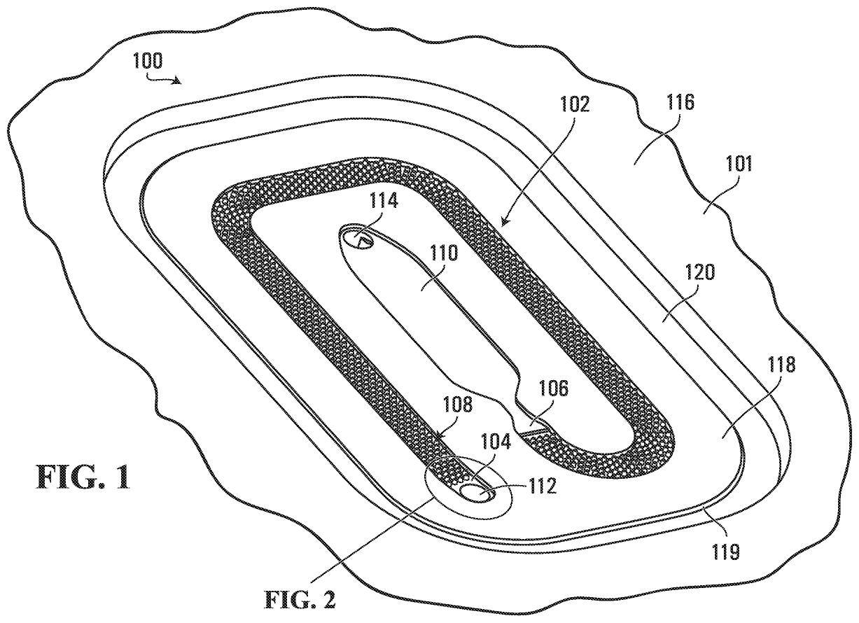

For example, FIG. 1 illustrates an isometric view of an example of a haemolysis stage 100 of a diagnostic consumable. The haemolysis stage 100 is implemented as part of a substrate 101 and includes a haemolysis channel 102 having an inlet port 104 for receiving whole blood, an outlet port 106 for dispensing haemolysed blood, and an array of micro-projections 108 that extend into the haemolysis channel to define a plurality of flow paths therebetween along a length of the haemolysis channel between the inlet port and the outlet port. The array of micro-projections 108 has disposed thereon a haemolytic reagent for interaction with the whole blood as the whole blood is flowed through the haemolysis channel 102 to generate haemolysed blood. The inlet port 104 and/or the outlet port of the haemolysis channel 102 may be fluidly connected to other fluidic channels or components on the substrate 101. For example, in the embodiment illustrated in FIG. 1, the inlet port 104 of the haemolysis channel 102 is fluidly connected to another fluidic channel or component on the substrate 101 through a via 112, and the outlet port 106 of the haemolysis channel is fluidly connected to a chamber 110, which is in turn fluidly connected to another fluidic channel or component on the substrate 101 through a via 114. For example, in some embodiments, the via 114 may be fluidly connected to a vacuum port downstream of the chamber 110 that is configured for application of a vacuum source through which a vacuum can be applied as a pumping force to pull a blood sample through the haemolysis channel 102 from the inlet port 104 to the outlet port 106 and into the chamber 110. For example, the vacuum source may be provided by a diagnostic device into which the diagnostic consumable is inserted or otherwise engaged. Such a vacuum port is merely one example of a fluid displacement element that may be incorporated into a diagnostic consumable in order to enable an external stimulus to be applied to the diagnostic consumable to pump a fluid sample through the channel. In the case of a downstream vacuum port, the external stimulus is in the form of a negative pressure or vacuum that pumps the fluid sample by pulling the fluid sample through the channel. In other implementations, the fluid displacement element may be a pumping port fluidly connected upstream of the channel and configured for application of a positive pressure source to push the fluid sample through the channel. In some embodiments, rather than directly applying a negative or positive pressure source to the diagnostic consumable, the external stimulus may be a mechanical or electrical stimulus that activates a pumping mechanism on the diagnostic consumable. For example, in some embodiments the fluid displacement element may be an air bladder that can be mechanically actuated in order to urge sample fluid through the channel. For example, the air bladder may have an exit port fluidly connected upstream of the channel through which air can be expelled from the air bladder by mechanically squeezing a flexible portion of the air bladder in order to push the fluid sample through the channel. Such an air bladder could be implemented by a cavity in the substrate 101 that is covered by a flexible cover layer affixed to an external surface of the substrate, for example. The mechanical stimulus to actuate/squeeze the air bladder could be provided by a mechanical actuator, e.g., an electrical motor, of a diagnostic device into which the diagnostic consumable is inserted or otherwise engaged.

The haemolysis channel 102 is illustrated in FIG. 1 as a channel with a generally rectangular cross-section and multiple turns along its length, however other geometries of a haemolysis channel are also possible. For example, in some embodiments a haemolysis channel could be substantially straight along its length. However, the inclusion of multiple turns or curves along the length of the channel may facilitate fitting a longer channel in a limited area on a diagnostic consumable.

Haemolysed blood dispensed into the chamber 110 may be analyzed. For example, in the example illustrated in FIG. 1, the chamber 110 is configured as a cuvette for an optical assay, such as co-oximetry.

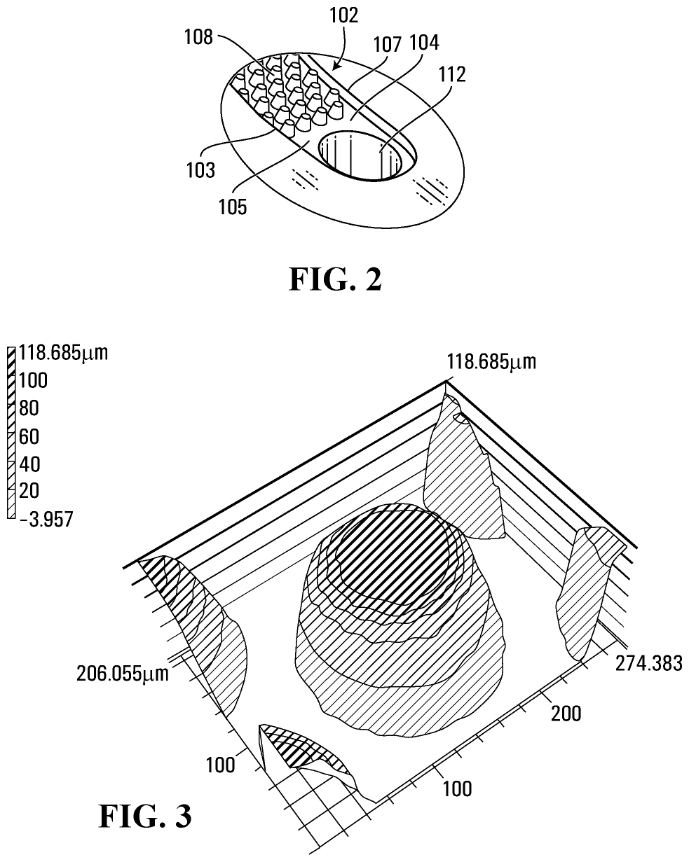

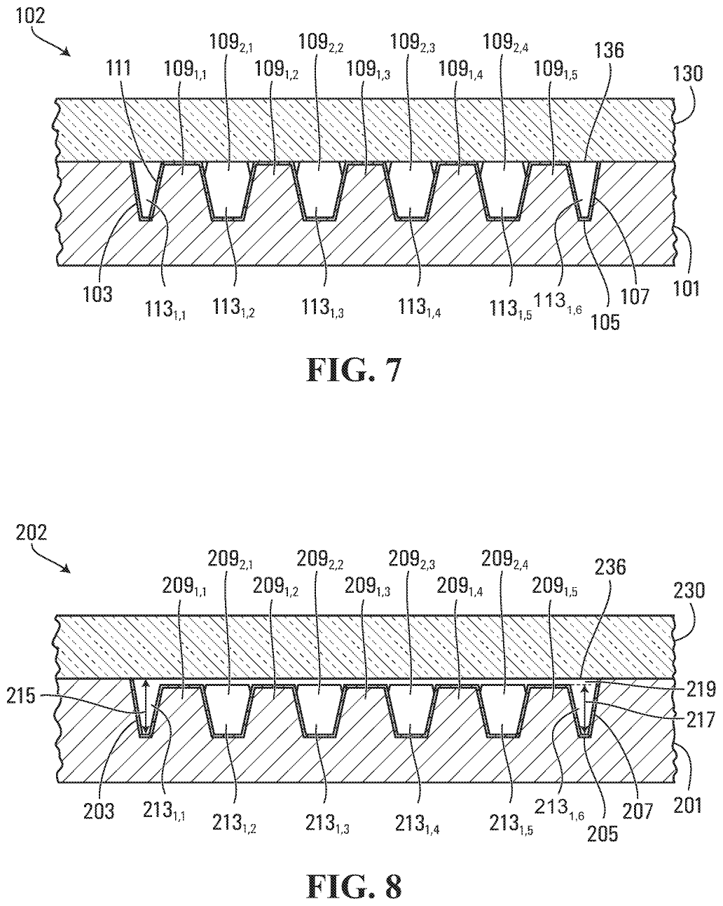

FIG. 2 is a magnified isometric view of a portion of the haemolysis channel 102 of FIG. 1 showing the disposition of the array of micro-projections 108 in the channel. In particular, FIG. 2 shows that the micro-projections in this example are disposed in staggered rows, each row being arranged substantially transverse to a direction of flow through the haemolysis channel from the inlet port 104 to the outlet port 106. Moreover, in this example, the micro-projections 108 are arranged with a generally uniform spacing therebetween. In addition, FIG. 2 shows that in this example the micro-projections 108 have the form of micro-pillars with a generally circular cross-sectional shape and a slight taper from bottom to top. In some embodiments the substrate 101 may be formed via injection moulding, and the slightly tapered shape of the micro-projections may facilitate removal of the substrate 101 from an injection mould. For example, FIG. 3 is a scanning confocal microscope image of a portion of the array of micro-projections 108 implemented on a plastic substrate obtained via a plastic injection moulding process. As shown in FIG. 3, each micro-projection has a generally circular cross-sectional shape and a slight taper from bottom to top to facilitate removal of the plastic substrate from the mould. For similar reasons, in some embodiments the side walls 103,107 of the haemolysis channel 102 may be slightly inclined outward from bottom to top, as discussed in further detail below with reference to FIG. 7.

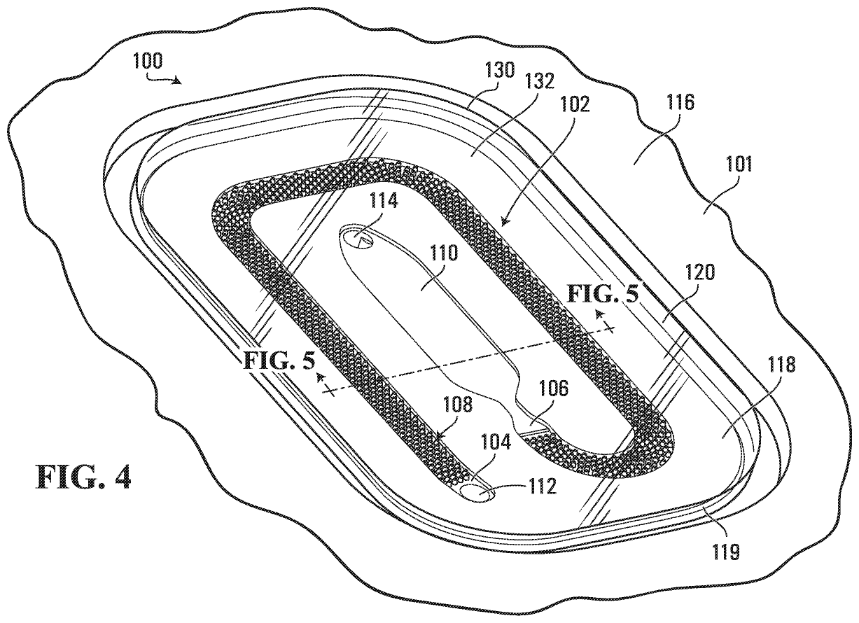

In the example shown in FIGS. 1 and 2, the haemolysis channel 102 has a bottom surface 105 and generally opposed side surfaces 103 and 107. In some embodiments, a top surface of the haemolysis channel 102 is formed by affixing a cover layer 130 to the substrate 101 so that the cover layer 130 covers the haemolysis channel 102. FIG. 4 shows an example of the haemolysis stage 100 of FIGS. 1 and 2 with a cover layer 130 affixed to the substrate 101. In the example shown in FIG. 4, the cover layer 130 covers the haemolysis channel 102 and the chamber 110. During manufacturing, the haemolysing reagent may be deposited and dried-down on the array of micro-projections 108 before the cover layer 130 is affixed to the substrate 101. For example, a solution of a haemolysing reagent, such as a surfactant/detergent, dissolved in water and isopropyl alcohol may be deposited on the array of micro-projections 108 and allowed to dry-down on the micro-pillars before the cover layer 130 is affixed to the substrate 101 to cover the haemolysis channel 102. The array of micro-projections 108 shown in FIGS. 1 to 4 has strong capillarity properties, which facilitates relatively even spreading of the haemolysing reagent solution throughout the array and the surfaces 103, 105 and 107 of the channel (except the top surface 136 formed by the cover layer 130, which may be affixed after dry-down of the reagent in some embodiments). In some embodiments, the haemolytic reagent solution is applied to the micro-pillars by dispensing a predefined number of drops of the haemolytic reagent solution onto the array and allowing the capillarity of the array to disperse the haemolytic reagent solution amongst micro-projections of the array. In addition, this capillarity property tends to retain the liquid reagent solution in the area of the channel 102 in which the array of micro-projections 108 is located, rather than flowing into the chamber 110, which could potentially affect an optical assay in the chamber 110.

The cover layer 130 may be optically transparent in order to facilitate optical measurement of haemolysed blood in the chamber 110. For example, the cover layer 130 may be made from a material with relatively high optical transparency, such as glass or polymethyl methacrylate (PMMA), also known as acrylic or acrylic glass.

In the example shown in FIGS. 1 to 4, the haemolysis stage 100 is formed in a “well” 120 in the substrate 101 so that, when the cover layer 130 is affixed to the substrate 101 to cover the haemolysis stage 100, a top surface 132 of the cover layer 130 is a short distance below a top surface 116 of the substrate 101 to avoid potential scratching/fouling of top surface 132 of cover layer 130 during subsequent manufacturing steps. For example, in the example illustrated in FIGS. 1 to 4, the haemolysis stage 100 is formed into a top surface 118 of an “island” 119 within the “well” 120, wherein when the cover layer 130 is affixed to the top surface 118 to cover the haemolysis stage 100, the top surface 132 of the cover layer 130 is slightly below the top surface 116 of the substrate 101. Other arrangements are possible. For example, in some embodiments the top surface 132 of the cover layer 130 may be substantially co-planar with the top surface 116 of the substrate 101, In other embodiments the top surface 132 of the cover layer 130 may be slightly above the top surface 116 of the substrate 101, The cover layer 130 may be affixed to the substrate 101 by any know affixing means. For example, in some embodiments the cover layer 130 is adhesively bonded to the substrate 101.

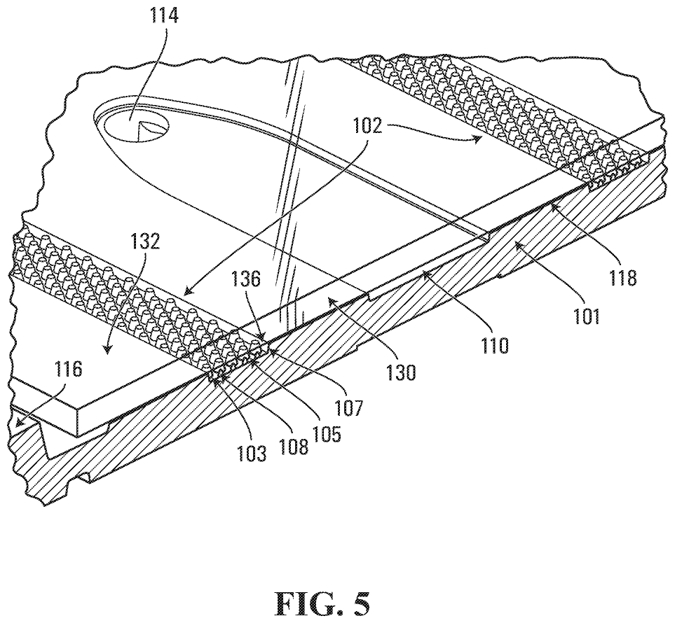

FIG. 5 is a cross-sectional view of the haemolysis stage 100 of FIG. 4, taken along the line illustrated in FIG. 4. As shown in FIG. 5, in this example the haemolysis channel 102 has a bottom surface 105, a top surface 136 generally opposed to the bottom surface 105, and generally opposed side surfaces 103,107 extending between the bottom surface 105 and the top surface 136, and the micro-projections 108 extend substantially the full height of the channel between the bottom surface 105 and the top surface 136. More generally, micro-projections may extend into a channel at least a portion of the height of the channel, and may extend from the top surface of the channel, the bottom surface of the channel, or in some cases from both the top and bottom surfaces of the channel, as discussed in further detail below with reference to FIGS. 7 to 10.

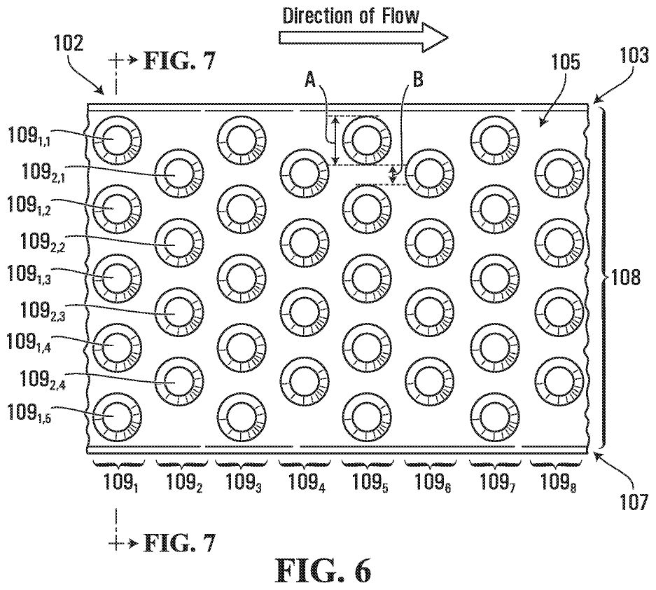

FIG. 6 is a plan view of a portion of the haemolysis channel 102 of the haemolysis stage 100 of FIG. 4. The portion of the haemolysis channel 102 shown in FIG. 6 includes eight staggered rows 109₁, 109₂, 109₃, 109₄, 109₅, 109₆, 109₇ and 109₈, respectively, of micro-projections. The staggered rows 109₁-109₈ of micro-projections are arranged in the channel 102 such that the micro-projections in each row are offset, in a direction transverse to the direction of flow through the haemolysis channel, relative to the micro-projections in the adjacent row(s). For example, the second row 109₂ of micro-projections, which includes four micro-projections 109_{2,1}, 109_{2,2}, 109_{2,3} and 109_{2,4}, is offset relative to the first row 109₁ of micro-projections, which includes five micro-projections 109_{1,1}, 109_{1,2}, 109_{1,3}, 109_{1,4} and 109_{1,5}, such that the micro-projections in the second row 109₂ are disposed substantially midway between the micro-projections in the first row 109₁. Furthermore, as shown in FIG. 6, every second row of micro-projections is substantially aligned in the direction of flow through the haemolysis channel 102. For example, the micro-projections in the third row 109₃ are substantially aligned, in the direction of flow through the haemolysis channel, with the micro-projections in the first row 109₁, and the micro-projections in the fourth row 109₄ are substantially aligned with the micro-projections in the second row 109₂, and so on. Moreover, in this example, the micro-projections 108 have a cross-sectional dimension A, measured transverse to the direction of flow through the haemolysis channel 102, that is greater than a separation distance B between adjacent micro-projections in each of the rows 109₁-109₈, which means that there are no straight flow paths through the array of micro-projections 108. Furthermore, the generally uniform spacing (separation distance B) between any two adjacent micro-pillars in each row means that as a blood cell flows through each row it is never more than one half of the separation distance B away from a reagent coated surface, thereby potentially resulting in a more consistent diffusion distance across the cross-section of the haemolysis channel 102. This is illustrated by way of example in FIG. 7, which is a a cross-sectional view of the haemolysis channel 102 of FIG. 6, taken along the line illustrated in FIG. 6 that extends through the first row 109₁ of micro-projections transverse to the direction of flow.

As shown in FIG. 7, in this example the surfaces of the haemolysis channel 102, with the exception of the top surface 136 formed by the bottom surface of the cover layer 130, are coated with dried-down haemolytic reagent 111. Moreover, from this view it can be seen that the sidewalls 103 and 107 of the haemolysis channel 102 and the five micro-projections 109_{1,1}-109_{1,5} define six flow paths 113_{1,1}, 113_{1,2}, 113_{1,3}, 113_{1,4}, 113_{1,5} and 113_{1,6} through which a blood cell may move along the haemolysing channel 102 as it traverses the first row 109₁ of micro-projections. As shown in FIGS. 6 and 7, the offset of the second row 109₂ of micro-projections relative to the first row 109₁ aligns the micro-projections 109_{2,1}-109_{2,4} of the second row with the flow paths 113_{1,2}-113_{1,5} defined by the first row. Furthermore, the generally uniform spacing of the micro-projections 108 in this example means that as a blood cell may never be more than one half of the generally uniform separation distance away from a reagent coated surface of a micro-projection as the blood cell is flowed through the array of micro-projections.

In some embodiments, the spacing between micro-projections could be selected based, at least in part, on a dimension of a component of the sample fluid that is to be flowed through the channel to interact with the reagent that has been dried-down in the channel. For example, in a haemolysis channel for the haemolysis of whole human blood, the spacing between micro-projections may be selected based, at least in part, on the typical size of a human red blood cell. A typical human red blood cell is generally disk-shaped and has a disk diameter of approximately 6-8 μm, a thickness at its thickest point of 2-2.5 μm and a minimum thickness in its centre of 0.8-1 μm. In some embodiments, the spacing between micro-projections may be on the order of approximately a multiple of 10 of the diameter of a typical red blood cell. For example, in some embodiments the spacing between micro-projections may be on the order of 60-100 μm. The 10 times multiple and the 60-100 μm spacing are merely non-limiting examples. Other multiples and dimensions are possible and are contemplated within the scope of the present disclosure.

As shown in FIG. 7, the generally opposed sidewalls 103,107 of the haemolysing channel 102 in this example are inclined outward, and the micro-projections have a slight taper from bottom to top, which, in those cases where the substrate is formed via moulding may facilitate removal of the substrate 101 from a mould.

The plurality of flow paths defined by the micro-projections within the haemolysis channel 102 means that the issue of high sample flow resistance associated with a single thin channel is substantially mitigated. Given its low flow resistance, the haemolysis channel 102 can be made long enough that blood emerges from it completely haemolysed even when delivered at high speed by an external pressure source, provided that the time-constant of reagent dissolution is sufficient. This is due to numerous small-distance diffusion events afforded by a series of micro-projections along the length of the array. Experimental results obtained with a haemolysis channel implemented according to the haemolysis channel 102 shown in FIGS. 1 to 7 demonstrated complete haemolysis in less than 5 seconds. In addition, it has been observed that when a blood sample is flowed through the haemolysis channel 102 it propagates with a substantially flat flow-front due to the array of micro-projections within the channel. In contrast, a blood sample flowed through channel without micro-projections typically propagates with a parabolic flow-front.

The structure of the haemolysis channel 102 of FIGS. 1 to 7 is provided by way of example. Other haemolysis channel structures could also or instead be used in a diagnostic consumable. For example, in the haemolysis channel 102 shown in FIGS. 4 to 7, the micro-projections 108 extend substantially the full height of the haemolysis channel 102 between the bottom surface 105 formed by the substrate 101 and the top surface 136 formed by the cover layer 130. However, as noted earlier, in other embodiments micro-projections may extend less than the full height of the channel, and may extend from the top surface of the channel, the bottom surface of the channel, or possibly both the top and bottom surfaces of the channel. Examples of such alternative embodiments are shown in FIGS. 8 to 10.

FIG. 8 is a cross-sectional view of another haemolysis channel 202, taken along a transverse line that extends through a first row of micro-projections transverse to the direction of flow along the channel. Similar to the haemolysis channel 102 shown in FIG. 7, the haemolysis channel 202 shown in FIG. 8 has a bottom surface 205 formed by a substrate 201, a top surface 236 formed by the bottom surface of a cover layer 230, and generally opposed side walls 203, 207 extending between the bottom surface 205 and the top surface 236. Moreover, from this view it can be seen that the first row of micro-projections includes five micro-projections 209_{1,1}, 209_{1,2}, 209_{1,3}, 209_{1,4} and 209_{1,5} defining six flow paths 213_{1,1}, 213_{1,2}, 213_{1,3}, 213_{1,4}, 213_{1,5} and 213_{1,6} through which a blood cell may move along the haemolysing channel 202 as it traverses the first row of micro-projections. From this view it can also be seen that a second row of micro-projections includes four micro-projections 209_{2,1}, 209_{2,2}, 209_{2,3} and 209_{2,4} that are offset relative to the micro-projections in the first row so that they are substantially aligned with the flow paths 213_{1,2}-213_{1,5} defined between the micro-projections of the first row. However, in this example, the height 217 of the micro-projections extends less than the full height 215 of the channel 202 so that there is a gap 219 between the top surface 236 of the channel and the micro-projections.

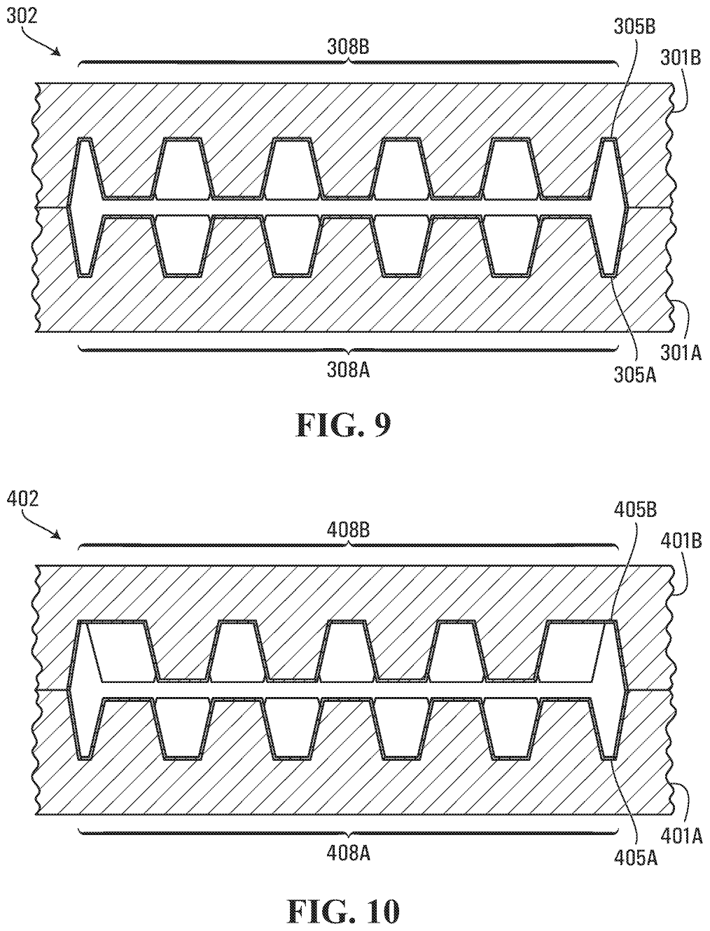

FIG. 9 is a cross-sectional view of yet another haemolysis channel 302, taken along a transverse line that extends through a first row of micro-projections transverse to the direction of flow along the channel. In this example, micro-projections extend from both a bottom surface 305A and a top surface 305B of the haemolysis channel 302. In particular, in this example, the bottom surface 305A of the channel 302 is formed by a first substrate 301A and the top surface 305B of the channel 302 is formed by a second substrate 301B. A first array of micro-projections 308A extends into the channel 302 from the bottom surface 305A, and a second array of micro-projections 308B extends into the channel 302 from the top surface 305B. Micro-projections in each of the first and second arrays 308A, 308B are arranged in staggered rows. In this example, each row of micro-projections in the first array of micro-projections 308A on the first substrate 301A is substantially aligned with a corresponding row of micro-projections in the second array of micro-projections 308B on the second substrate 301A.

An alternative arrangement of micro-projections in a haemolysis channel 402, in which micro-projections extend from both a bottom surface 405A and a top surface 405B of the haemolysis channel 402, is shown in FIG. 10. In this example, each row of micro-projections in a first array of micro-projections 408A on a first substrate 401A is offset relative to a corresponding row of micro-projections in a second array of micro-projections 408B on a second substrate 401B. In particular, in this arrangement, micro-projections in the first array of micro-projections 408A on the first substrate 401A extend toward spaces between micro-projections in the second array of micro-projections 408B on the second substrate 401B and vice versa.

In the embodiments shown in FIGS. 6 to 10, the first and last micro-projection in each row of micro-projections are spaced apart from the sidewalls 103 and 107 of the haemolysis channel 102, and this spacing serves as a potential flow path. For example, referring again to FIGS. 6 and 7, in the first row 109₁ it can be seen that the flow path 113_{1,1} is defined between the sidewall 103 and the first micro-projection 109_{1,1}, while the flow path 113_{1,5} is defined between the sidewall 107 and the fifth micro-projection 109_{1,5}. Similarly, in the second row 109₂ there is a first flow path defined between the sidewall 103 and the first micro-projection 109_{2,1}, while a fifth flow path is defined between the sidewall 107 and the fifth micro-projection 109_{2,5}. As can be seen in FIG. 6, the micro-projections 109 in this embodiment are distributed such that, in each of the odd rows 109₁, 109₃, 109₅, 109₇ the spacing between the outermost micro-projections of the row and the sidewalls 103 and 107 of the channel 102 is less than the separation distance between the micro-projections in that row, while in each of the even rows 109₂, 109₄, 109₆, 109₈ the spacing between the outermost micro-projections of the row and the sidewalls of the channel is greater than the separation distance between the micro-projections in that row. For example, the spacing that defines the flow path 113_{1,1} between the sidewall 103 and the first micro-projection 109_{1,1} in the first row 109₁ is less than the separation distance between adjacent micro-projections in the first row, while the spacing that defines a flow path between the sidewall 103 and the first micro-projection 109_{2,1} in the second row 109₂ is greater than the separation distance between adjacent micro-projections in the second row. This may cause the flow pattern to be less uniform near the sidewalls 103 and 107. However, although the flow paths between the outermost micro-projections and the sidewalls of the channel may at some points be larger than the separation distance between adjacent micro-projections, in general the larger flow path widths are not so large that the cumulative reagent-sample interaction there becomes insufficient. For example, in some embodiments the maximum spacing defining flow paths between the outermost micro-projections and the sidewalls of the channel may be no more than 200% of the separation distance between adjacent micro-projections, in some embodiments no more than 175% of the separation distance between adjacent micro-projections, in some embodiments no more than 150% of the separation distance between adjacent micro-projections, in some embodiments no more than 125% of the separation distance between adjacent micro-projections or even less.

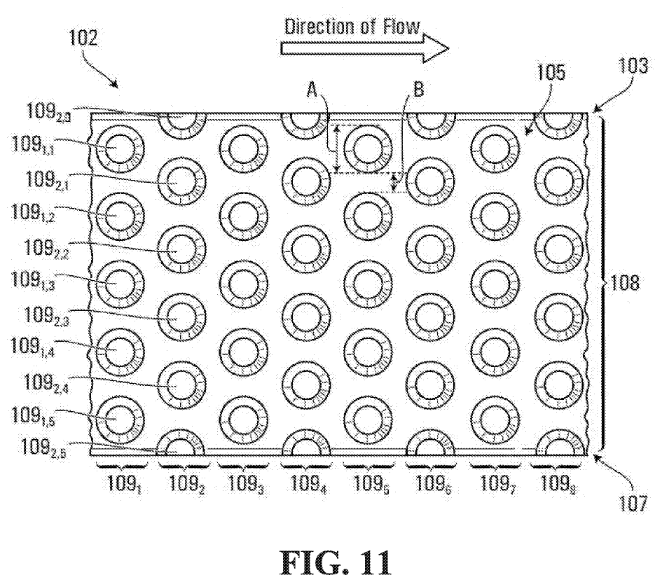

In other embodiments, there may be no spacing between a micro-projection and a sidewall of a channel. For example, in some embodiments a micro-projection or at least a portion thereof may form part of one or more of the sidewalls of a channel. An example of such an embodiment is shown in FIG. 11, which shows a haemolysis channel 102 that differs from the haemolysis channel 102 shown in FIG. 6 in that every second row 109₂, 109₄, 109₆, 109₈, etc. additionally includes a first partial micro-projection formed as part of the first sidewall 103 and a second partial micro-projection formed as part of the second sidewall 107. For example, in addition to the four micro-projections 109_{2,1}-109_{2,4} of the second row 109₂ of micro-projections in FIG. 6, the second row 109₂ of micro-projections in FIG. 11 further includes a first partial micro-projection 109_{2,0} and a second partial micro-projection 109_{2,5}. The inclusion of the partial micro-projections at the sidewalls potentially makes the flow pattern and the sample-reagent interaction distance more uniform near the sidewalls of the channel, but may negatively impact manufacturability. For example, it may be more difficult to reliably manufacture such an embodiment repeatedly via injection molding.

Although the example embodiment described above and shown in FIGS. 1 to 11 has been described as a haemolysis stage for haemolysing a whole blood sample, the same or similar structure could be used for other fluid sample preparation functions. For example, the same structure could be used to mix a whole blood sample with a coagulant for an activated clotting time (ACT) test e.g., by changing the material that is deposited on the array of micro-projections from a haemolytic reagent to a coagulant. In such cases, clotting of the resulting mix of whole blood and coagulant in the chamber 110 could be measured optically using an optical source and sensor external to the diagnostic device and/or or by means of one or more electrochemical sensors located somewhere downstream of the channel 102.

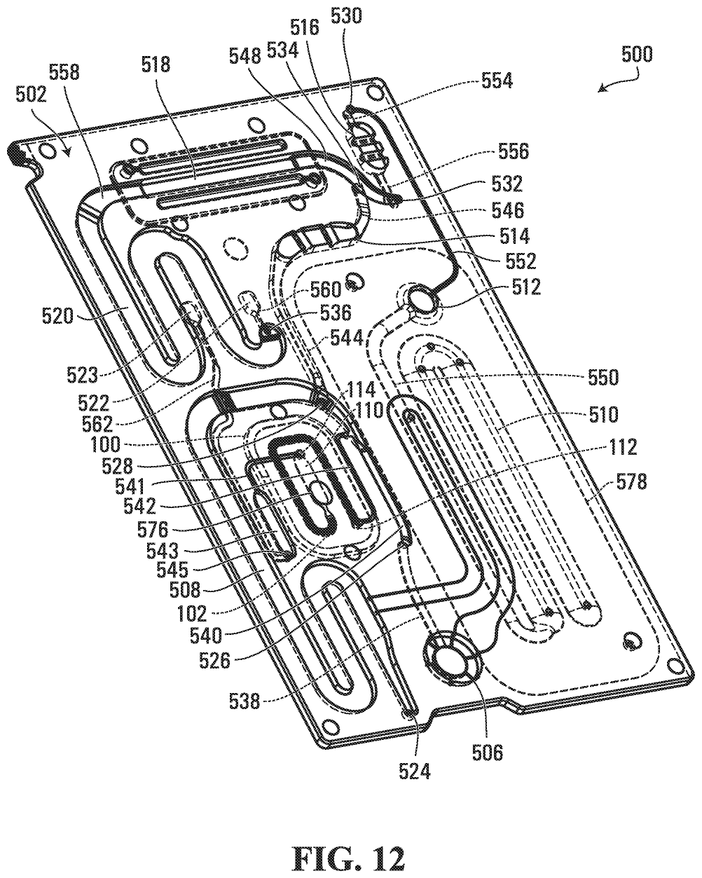

A non-limiting example of a substrate and diagnostic consumable incorporating the haemolysis stage of FIGS. 1 to 7 will now be described with reference to FIGS. 12 to 18. It is to be understood that this example implementation is provided for illustrative purposes only, and that other implementations and configurations of the haemolysis stage, the substrate and/or the diagnostic consumable are possible and are contemplated within the present disclosure.

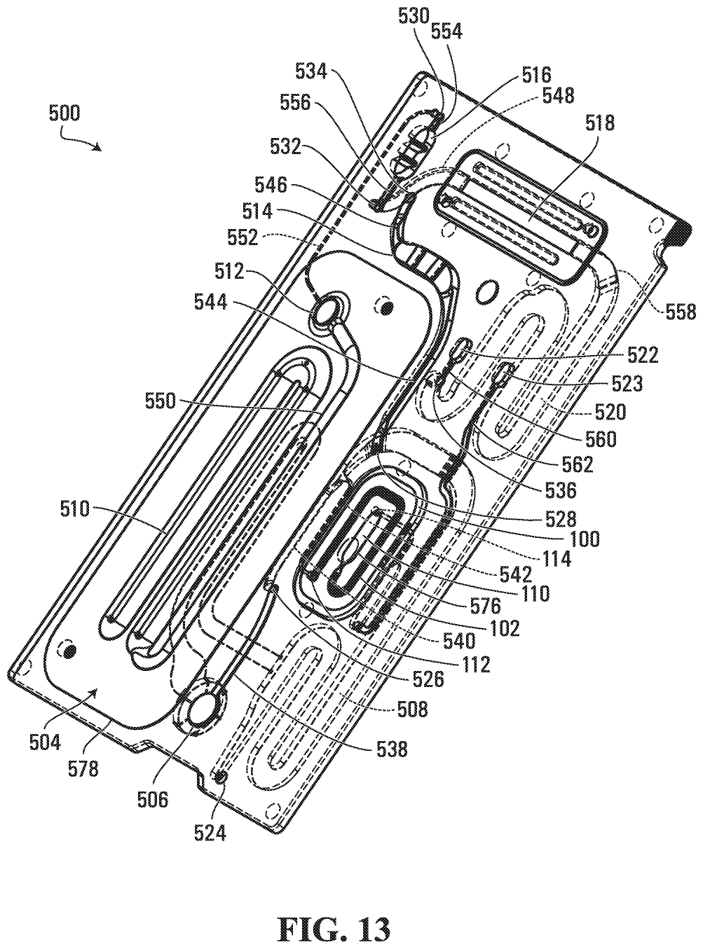

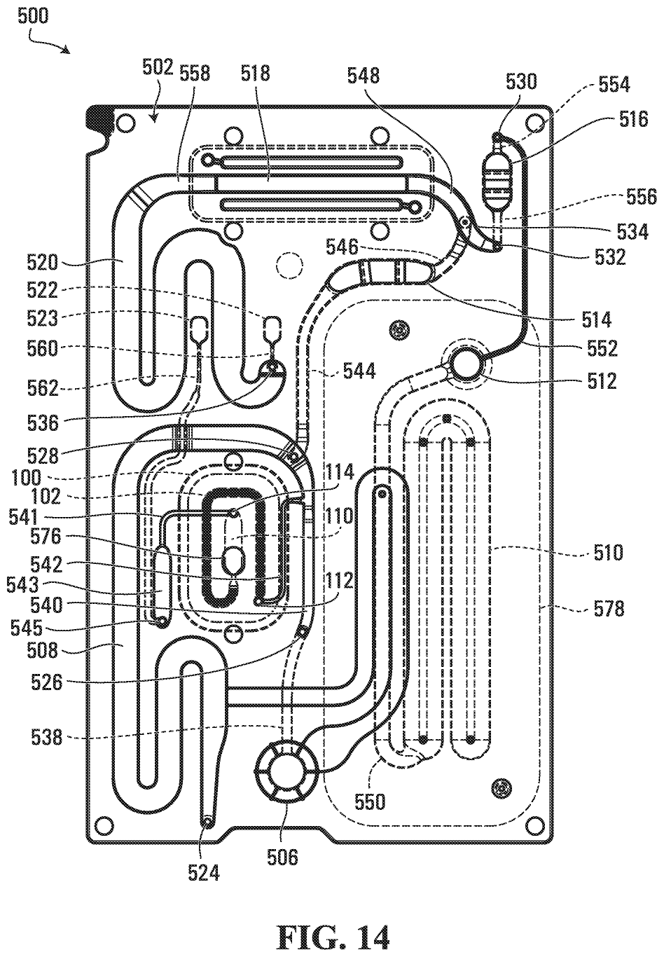

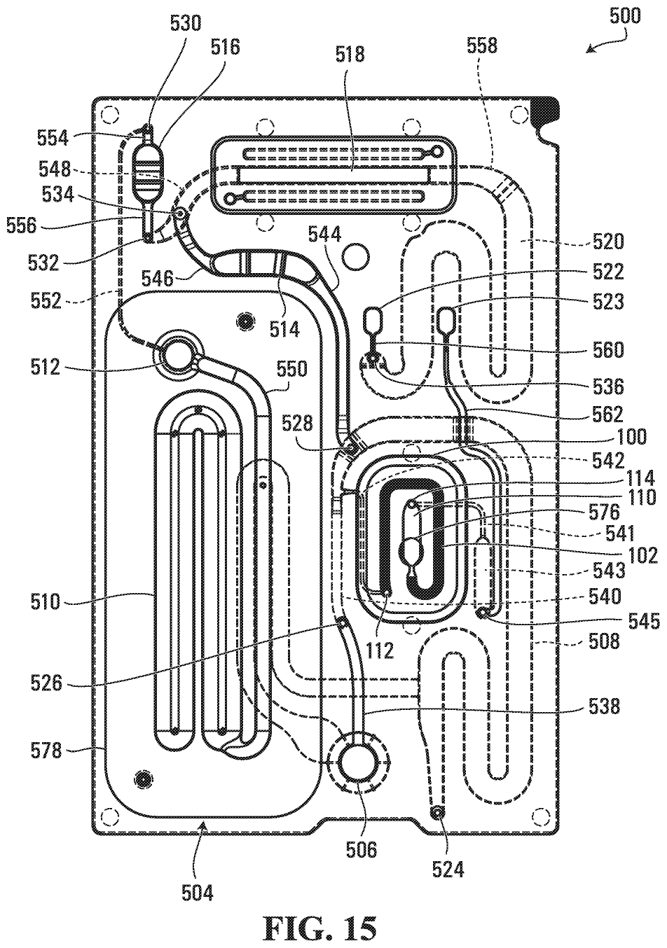

FIGS. 12 to 15 illustrate an example substrate 500 for a diagnostic consumable that includes multiple sensing regions. FIGS. 12 and 13 are isometric views of the substrate 500, and FIGS. 14 and 15 are plan views of the substrate. FIGS. 12 and 14 are views of a top surface 502 of the substrate 500, and FIGS. 13 and 15 are views of a bottom surface 504 of the substrate. The terms “top” and “bottom” are used herein for ease of reference only, and do not require or imply a certain orientation of the substrate 500. Although the substrate 500 could be designed to be operated with the top surface 502 facing vertically upwards and the bottom surface 504 facing vertically downwards, this might not be the case in all implementations. Moreover, the orientation of the top surface 502 and the bottom surface 504 of the substrate 500 could have minimal or no impact on fabrication, storage and/or transportation of the substrate.

The substrate 500 is illustrated as being a rectangular prism that is approximately the size and shape of a credit card, but this is only an example. The substrate 500 could also or instead be other shapes such as triangular or circular, for example. The substrate 500 could be made out of plastics, ceramics, glass and/or metal, for example. The substrate 500 could be a single, unitary body or part. The dimensions of the substrate 500 are not limited to any specific ranges or values. The length and width of the substrate 500 could be considered to define the area of the top surface 502 and the bottom surface 504. In some implementations, the length and/or width of the substrate 500 is on the order of centimeters. In some implementations, the length and/or width of the substrate 500 is on the order of millimeters. Other lengths and/or widths of the substrate 500 are also possible. The thickness of the substrate 500 could be measured as the distance between the top surface 502 and the bottom surface 504 of the substrate. In some implementations, the thickness of the substrate 500 is on the order of centimeters. In some implementations, the thickness of the substrate 500 is on the order of millimeters. In some implementations, the thickness of the substrate 500 is on the order of micrometers. Other thicknesses of the substrate 500 are also possible. Although the top surface 502 and the bottom surface 504 of the substrate 500 are illustrated as being substantially flat, this might not be the case in all embodiments. For example, the top surface and/or the bottom surface of a substrate could also or instead be triangular, conical and/or hemispherical in shape. Accordingly, the thickness of a substrate could vary along its length and/or width. The substrate 500 is illustrated as being transparent, however substrates could also or instead be, in whole or in part, translucent or opaque.

The substrate 500 includes the haemolysis stage 100 of FIGS. 1 to 7, in which a portion of the chamber 110 functions as an optical sensing region 576. The substrate 500 further includes a sample fluid input port 506, a sample fluid reservoir 508, a fluid reservoir 510, a valve hole 512, two bubble traps 514, 516, another sensing region 518, waste fluid reservoirs 520, 543, multiple pump connection ports 522, 523, multiple vias 112, 114, 524, 526, 528, 530, 532, 534, 536, 545, and multiple channels 538, 540, 541, 542, 544, 546, 548, 550, 552, 554, 556, 558, 560, 562. In FIGS. 12 to 15, solid lines are used to illustrate components that are directly in view in each figure, and dashed lines are used to illustrate components that are hidden from view by at least a portion of the substrate 500.

The channels 538, 540, 541, 542, 544, 546, 548, 550, 552, 554, 556, 558, 560, 562 are provided to carry one or more fluids in the substrate 100. The channels 540, 541, 542, 548, 552, 558 are trenches or grooves in the top surface 502 of the substrate 500. The channels 540, 541, 542, 548, 552, 558 are illustrated as being open at the top surface 502 of the substrate 500 in FIGS. 12 and 14. Similarly, the channels 538, 544, 546, 550, 554, 556, 560, 562 are trenches or grooves in the bottom surface 504 of the substrate 500, which are open at the bottom surface of the substrate in FIGS. 13 and 15. Any or all of the channels 538, 540, 541, 542, 544, 546, 548, 550, 552, 554, 556, 558, 560, 562 could be microfluidic channels. For example, the width and/or height of any or all of the channels 538, 540, 541, 542, 544, 546, 548, 550, 552, 554, 556, 558, 560, 562 could be on the order of micrometers. The width and/or height of any or all of the channels 538, 540, 541, 542, 544, 546, 548, 550, 552, 554, 556, 558, 560, 562 could also or instead be on the order of millimeters or centimeters. The cross-sectional area of a channel or other fluidic component is generally measured as an area inside of the channel that is perpendicular to a direction of fluid flow. Although the channels 538, 540, 541, 542, 544, 546, 548, 550, 552, 554, 556, 558, 560, 562 are illustrated with generally rectangular cross-sections in FIGS. 12 to 15, one or more of these channels could have other cross-sectional shapes as well, such as semicircular or triangular, for example.

The vias 112, 114, 524, 526, 528, 530, 532, 534, 536, 545 are through-holes or bores that extend through the substrate 500. Vias could be used to fluidly connect two or more components of the substrate 500. For example, via 112 fluidly connects channel 542 and the haemolysis channel 102, via 114 fluidly connects chamber 110 and channel 541, via 526 fluidly connects channel 538 and channel 540, via 528 fluidly connects channel 540 and channel 544, via 530 fluidly connects channel 552 and channel 554, via 532 fluidly connects channel 548 and channel 556, via 534 fluidly connects channel 546 and channel 548, via 536 fluidly connects channel 560 and the waste fluid reservoir 520, and via 545 fluidly connects channel 562 and the waste fluid reservoir 543. Vias could also or instead be used to fluidly connect a component of the substrate 500 to the top surface 502 and/or bottom surface 504 of the substrate. For example, the via 524 fluidly connects the sample fluid reservoir 508 to the bottom surface 504 of the substrate 500. Although illustrated as circular holes, the vias could also or instead be other shapes such as rectangular or triangular, for example. The diameter of the vias could be similar to the width of one or more of the components that each via connects. For example, the diameter of the via 526 could be similar to the width of the channel 538 and/or the channel 540. However, the diameter of the vias could be different from the width of the components that each via connects.

The sample fluid input port 506 is provided to deliver a blood sample to the substrate 500. The sample fluid input port 506 is a conical or cylindrical opening in the top surface 502 of the substrate 500. The sample input port 506 is coupled to the channel 538. The sample input port 506 could be sized and shaped to engage with an end of a blood sample delivery device, such as a syringe or capillary tube (not shown), that delivers the blood sample. For example, in the case of a syringe, this engagement between the sample input port 506 and the syringe could form a seal such that, when the blood sample is propelled or pumped out of the syringe, the blood sample is forced into the channel 538 and does not spill out of the sample input port. In some embodiments, a gasket component is installed in the sample input port 506 in order to facilitate the sealing engagement with the sample delivery device.

The sample fluid reservoir 508 could be a relatively wide and long channel or chamber that is coupled to the channel 540. The sample fluid reservoir 508 is illustrated with a rectangular cross-section, however other cross-sectional shapes are also possible. The sample fluid reservoir 508 could be provided to store a blood sample after it is delivered into the substrate 500. The via 524 could act as an air vent to allow air to escape the sample fluid reservoir 508 when it is displaced by the addition of blood sample. During operation, the blood sample might stay in the sample fluid reservoir 508 for an amount of time that is on the order of milliseconds, seconds, or minutes, for example.

The fluid reservoir 510 could be a relatively wide and long channel or chamber that is coupled to the channel 550. The fluid reservoir 510 is illustrated as a U-shaped channel with a semicircular cross-section, however other geometries are also possible. In some embodiments the fluid reservoir 510 could be provided to store a calibration fluid or a wash fluid and/or a fluid pack that seals the calibration fluid or the wash fluid. The fluid pack could be positioned in a shallow depression provided by the fluid pack region 578. In embodiments where the fluid reservoir 510 stores a calibration fluid, the calibration fluid could be used to calibrate one or more sensors included on and/or coupled to the substrate 500. Calibration fluids could include fluids with known concentrations of one or more analytes. These analytes could correspond to analytes in the blood sample that might be measured using the substrate 500. In embodiments where the fluid reservoir 510 stores a wash fluid, the wash fluid could be used to wash one or more regions of the substrate 500. For example, the wash fluid could be used to wash away unbound components from an antigen-antibody interaction region.

The valve hole 512 could be a via or bore that extends through the thickness of the substrate 500. The channel 550 and the channel 552 could be fluidly connected by the valve hole 512. The valve hole 512 could be sized and shaped to accommodate and/or couple to a valve (not shown). This valve could control the flow of fluid from the channel 550 to the channel 552. When the valve is closed, the flow of fluid between the channel 550 and the channel 552 could be blocked. When the valve is opened, the flow of fluid between the channel 550 and the channel 552 could be permitted. In some implementations, the valve could be closed until a seal in the valve is ruptured, allowing fluid to flow into the channel 552.

The two bubble traps 514, 516 are provided to inhibit the movement of bubbles in the substrate 500. Each bubble that enters either of the bubble traps 514, 516 could be prevented from moving further downstream by one or more barriers in the bubble trap. Thus, the fluid that leaves the bubble traps 514, 516 could be free of air bubbles. The bubble trap 514 fluidly connects the channels 544, 546, and the bubble trap 516 fluidly connects the channels 554, 556.

The sensing region 518 includes a channel that is coupled to the channel 548 and to the channel 558. The sensing region 518 extends through the thickness of the substrate 500, and is therefore illustrated as being open at the top surface 502 and bottom surface 504 of the substrate in FIGS. 12 to 15. The sensing region 518 could include and/or be coupled to one or more sensors that measure properties of fluids in the sensing region. For example, the sensors could measure the concentration of one or more analytes in a fluid that flows from the channel 548 to the channel 558. The sensing region 518 could also or instead be referred to as an assay region.

The waste fluid reservoir 520 is fluidly coupled to the channel 558, and stores fluid that has flowed through the sensing region 518. The waste fluid reservoir 520 is illustrated in FIGS. 12 to 15 as a meandering channel with a rectangular cross-section, however other geometries of the waste fluid reservoir 520 are also possible.

The pump connection ports 522, 523 provide a connection to one or more external pumping systems. For example, these pumping systems could be provided in a diagnostic consumable reader module. The channel 560 is fluidly connected to the pump connection port 522, and the channel 562 is fluidly connected to the pump connection port 523. The pumping systems could include channels or tubes that fluidly connect to the pump connection ports 522, 523. In some embodiments, the pumping systems could include vacuum pumping systems that pull fluid in one or more channels of the substrate 500 towards the pump connection ports 522, 523.

The optical sensing or assay region 576 provides another sensing functionality to a diagnostic consumable incorporating the substrate 500. The channel 542 fluidly connects the channel 540 to the haemolysis channel 102 through via 112. The haemolysis channel 102 is fluidly connected to the chamber 110 within the optical sensing region 576. The channel 541 fluidly connects the chamber 110 and the waste fluid reservoir 543 through via 545. The channel 562 fluidly connects the waste fluid reservoir 543 to the pump connection port 523 through via 114. In operation, at least a portion of a blood sample could be directed through the channel 542, the haemolysis channel 102 and into the chamber 110 to be optically analyzed in the optical sensing region 576.

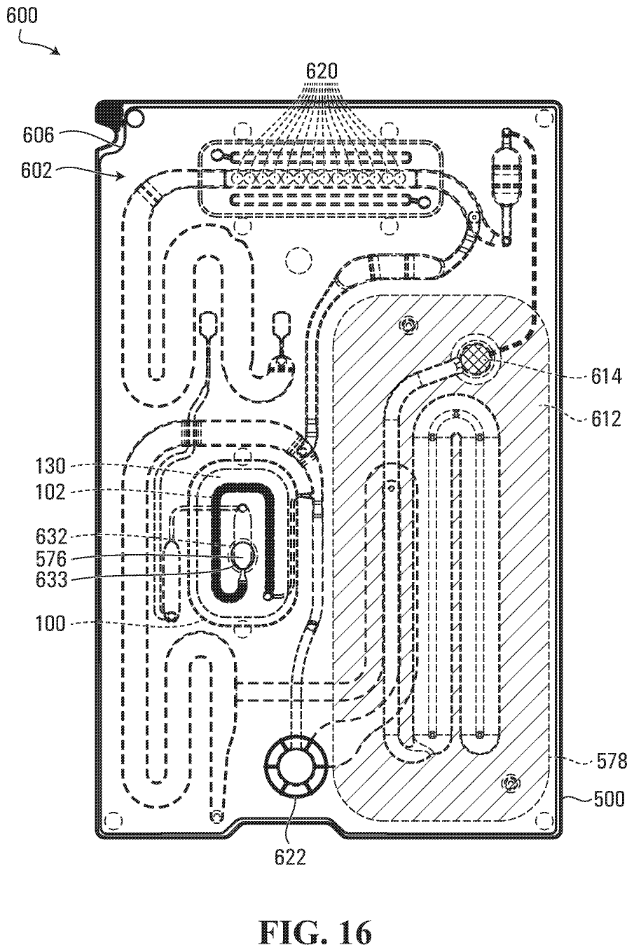

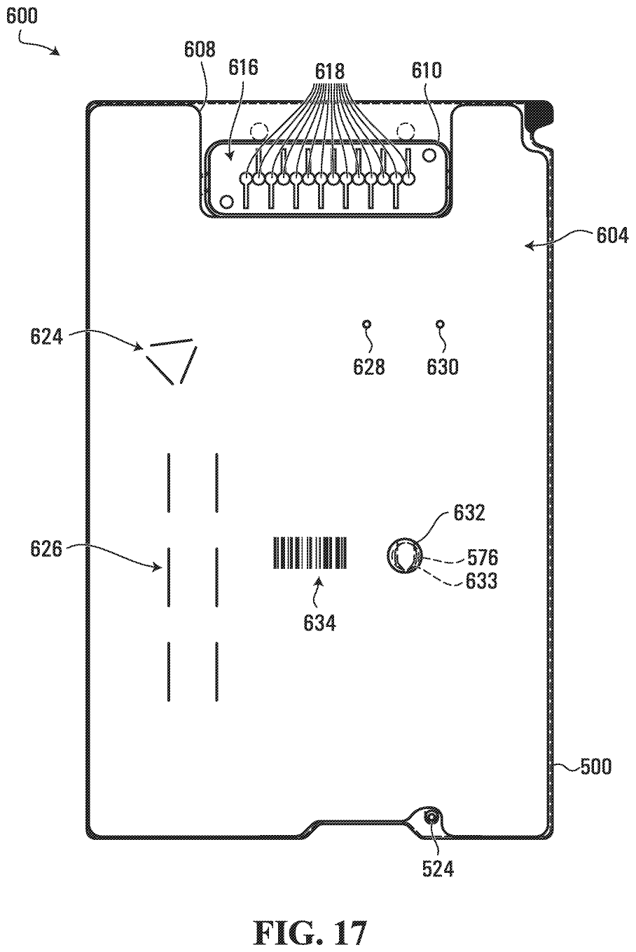

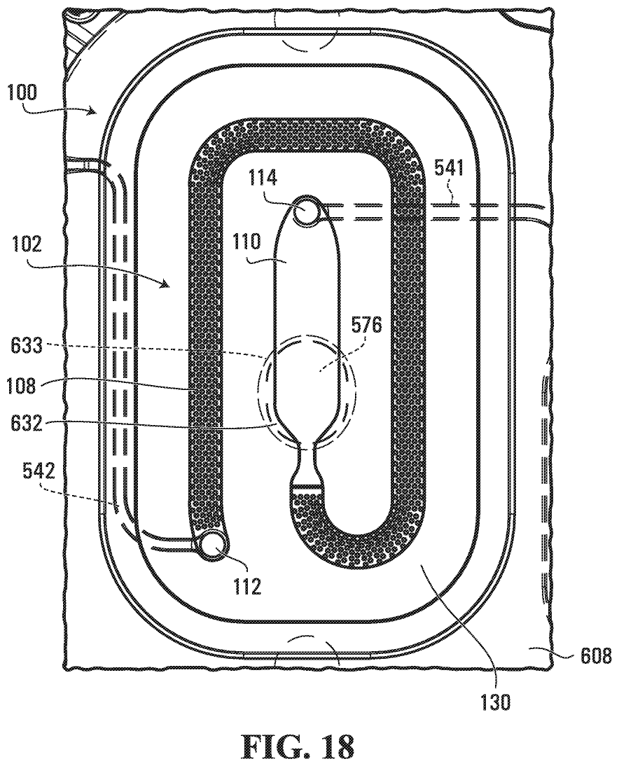

FIGS. 16 and 17 illustrate plan views of an example diagnostic consumable 600 that incorporates the substrate 500 shown in FIGS. 12 to 15. FIG. 18 is a plan view of the haemolysis stage 100 of the diagnostic consumable 600 shown in FIGS. 16 and 17. The diagnostic consumable 600 could be considered an assembled diagnostic card or test card for blood analysis and/or testing. In some implementations, the diagnostic consumable 600 is a microfluidic device. The diagnostic consumable 600 could be configured, by being sized and shaped for example, to be received by a diagnostic consumable reader module (not shown). FIG. 16 is a view of the top surface 602 of the diagnostic consumable 600, and FIG. 17 is a view of the bottom surface 604 of the diagnostic consumable. In addition to the substrate 500, the device 600 includes the cover layer 130 covering the haemolysis stage 100, a top cover layer 606, a bottom cover layer 608, a sensor array 610, a calibration fluid pack 612 (illustrated using parallel hatching) and a valve 614 (illustrated using cross-hatching). Many components of the substrate 500 are not labelled in FIGS. 16 and 17 for the purpose of clarity.

As described earlier, a haemolytic reagent can be deposited and dried-down on the array of micro-projections 108 in the haemolysis channel 102 before the cover layer 130 is affixed to the substrate 500. In this example, the cover layer 130 is transparent to facilitate optical sensing within the chamber 110 downstream of the haemolysis channel. In other embodiments, a cover layer for a haemolysis stage could be transparent, translucent, opaque, or a combination thereof.

At least a portion of the top surface 502 and bottom surface 504 of the substrate 500 are sealed using the top cover layer 606 and the bottom cover layer 608, respectively. The top and bottom cover layers 606, 608 could be impermeable to liquids (and possibly gases) to provide a liquid tight (and possibly gas tight) seal. In some implementations, the top and bottom cover layers 606, 608 could include plastic, metal and/or ceramic films that are bonded to the substrate 500 using an adhesive. For example, in some implementations, the top cover layer 606 and/or the bottom cover layer 608 could be implemented as an adhesive label or sticker. Non-limiting examples of adhesives include acrylic adhesives and silicone adhesives. The top and bottom cover layers 606, 608 could form a seal around one or more components of the substrate 600. For example, the top cover layer 606 could seal, at least in part, the sample fluid reservoir 508, the bubble traps 514, 516, the sensing region 518, the waste fluid reservoir 520 and the channels 540, 541, 542, 548, 552, 558. The bottom cover layer 608 could seal, at least in part, the sample input port 506, the fluid reservoir 510, the bubble traps 514, 516 and the channels 538, 544, 546, 550, 554, 556, 560, 562. The top cover layer 606 is illustrated as being substantially transparent and the bottom cover layer 608 is illustrated as being substantially opaque, but this is only an example. In general, either or both of the top cover layer 606 and the bottom cover layer 608 could be transparent, translucent, opaque, or a combination thereof. In FIG. 16, dashed lines are used to illustrate components that are under the top cover layer 606.

In this example, the sensor array 610, which could also be referred to as an electrode module, is bonded to the bottom surface 504 of the substrate 500. The sensor array 610 overlaps and seals at least a portion of the sensing region 518. The bottom cover layer 608 does not overlap the sensor array 610. The sensor array 610 could be fabricated using smart-card chip-module technology. In this example, the sensor array 610 includes a gold coated copper metal foil laminated to an epoxy foil element 616 with an optional adhesive. The metal foil is formed into an array of electrode elements 618. Each electrode element 618 could have a connection end for forming an electrical connection to a measuring circuit in a consumable reader module, for example. The connection ends of the electrode elements 618 are not labelled for reasons of clarity. Multiple sensors 620 are coupled to the electrode elements 618. Each of the sensors 620 are positioned over the sensing region 518 of the substrate 500. In use, the sensors 620 could be used to measure one or more properties of a calibration fluid and/or sample fluid in the sensing region 518. The sensors 620 could be electrochemical sensors that are used for measuring concentrations of gases, electrolytes and/or metabolites. The sensors 620 could include potentiometric sensors to measure sodium, potassium, ionized calcium, chloride, urea, TCO₂, pH levels and/or CO₂ partial pressure; amperometric sensors to measure O₂ partial pressure, glucose, creatinine, and/or lactate; and/or conductometric sensors to measure hematocrit, for example. The number and geometry of the electrodes 618 and the sensors 620 is provided by way of example only. The same module fabrication technology can be used to make sensor arrays with many different electrode/sensor numbers and geometries.

The calibration fluid pack 612 is sandwiched between the calibration fluid pack region 578 of the substrate 500 and the bottom cover layer 608. The calibration fluid pack 612 could fill the fluid reservoir 510 and the channel 550. The calibration fluid pack 612 could be provided to seal and store a calibration fluid, in order to improve the stability of the calibration fluid over time. For example, the calibration fluid pack 612 could inhibit gases, such as carbon dioxide, from permeating into and/or out of the calibration fluid.

The top surface 502 of the substrate 500 is substantially sealed by the top cover layer 606, with the exception of a hole 622 that corresponds to the location of the sample input port 506. The hole 622 allows a blood sample delivery device, such as a syringe or capillary tube, to be coupled to the sample input port 506 to deliver a blood sample into the diagnostic consumable 600. In addition, the top cover layer 606 also includes a second hole 633 that corresponds to the location of the optical sensing region 576. As discussed earlier, the sample input port 506 may include a gasket component that facilitates a sealing engagement between the sample input port 506 and the sample delivery device. For example, the gasket component may be a rubber or silicone component installed in the sample input port 506 and sized and shaped to sealingly engage a sample delivery device.

The bottom surface 504 of the substrate 500 is substantially covered by the bottom cover layer 608, with the exception that the sensor array 610 and the via 524 are not sealed by the bottom cover layer. The bottom cover layer 608 includes cuts or scoring 624, 626. The scoring 624, 626 could be provided to render the bottom cover layer 608 more malleable and workable in the area proximate the scoring. The position of the scoring 624 corresponds to the position of the valve 614. The scoring 624 could make the portion of the bottom cover layer 608 that is adjacent to the valve 614 more flexible, and could therefore permit the valve to be manipulated more easily. The position of the scoring 626 corresponds to the position of the fluid reservoir 510. The scoring 626 could make the portion of the bottom cover layer 608 adjacent to the fluid reservoir 510 more flexible, and therefore permit the calibration fluid pack 612 to be manipulated more easily. The bottom cover layer 608 also includes pump holes 628, 630 corresponding to the location of the pump connection ports 522, 523 on the substrate 500. The pump connection ports 522, 523 could be connected to a pump in a card reader module through the pump holes 628, 630. The pump holes 628, 630 could be sized and shaped to form a seal between the pump and the pump connection ports 522, 523. The bottom cover layer 608 overlaps the cover layer 130 of the haemolysis stage 100, but includes a hole 632 corresponding to the optical sensing region 576 and generally aligned with the hole 633 in the top cover layer 606. The holes 632, 633 and the transparency of the substrate 500 and the cover layer 130 in the area of the optical sensing region 576 facilitate optical sensing within the optical sensing region.

In this example, a 1D barcode 634 is printed on the bottom cover layer 608. The barcode 634 could be read by a card reader module when the diagnostic consumable 600 is inserted into the card reader module. The barcode 634 could authenticate the diagnostic consumable 600 and/or provide information regarding the diagnostic consumable. For example, the barcode 634 could indicate the date that the diagnostic consumable 600 was manufactured. The barcode 634 is one example of a machine-readable code that could be present on the bottom cover layer 608 or elsewhere on the diagnostic consumable. Other examples of machine-readable codes include 2D barcodes. Radio-frequency identification (RFID) chips or tags could also or instead be used.

In some embodiments, the diagnostic consumable 600 could be operated as follows. First, the diagnostic consumable 600 could be inserted into a corresponding slot of a diagnostic module, such as a portable or bench-top diagnostic card reader module. The diagnostic module might scan the barcode 634 to authenticate the diagnostic consumable 600. Second, the calibration fluid that is stored in the calibration fluid pack 612 could be propelled or pumped into the sensing region 618. This step could include the diagnostic module using a first actuator element to manipulate the valve 614 by pushing on the bottom cover layer 608 in an area proximate the scoring 624. The manipulation of the valve 614 could cause the plug in the valve to rupture, which opens the valve. At least a portion of the calibration fluid could then be pushed or pumped out of the calibration fluid pack 612, through the channel 550, the valve 512, the channel 552, the via 530, the channel 554, the bubble trap 516, the channel 556, the via 532, the channel 548, and into the sensing region 518. Pushing the calibration fluid out of the calibration fluid pack 612 could be performed by compressing the bottom cover layer 608 in the area proximate the scoring 626 using a second actuator element, such as a plunger, in the diagnostic module. When the calibration fluid is in the sensing region 518, it might be in contact with one or more of the sensors 620. The diagnostic module could include circuitry to contact the electrodes 618, which return measurements of the calibration fluid from the sensors 620. These measurements could be used to calibrate the diagnostic module for the diagnostic consumable 600, and thereby compensate for variations between different diagnostic consumables. The first and second actuator elements could be controlled by a motor-driven system in the diagnostic module. The diagnostic module could also include a form of temperature control, such as a heater in contact with the sensor array 610, to adjust the temperature of a fluid in the sensing region 518 and/or a heater in contact with, or proximal to, the optical sensing region 576, to adjust the temperature of a fluid in the optical sensing region. This temperature control could help provide consistency in the measurements made by the sensors 620 in the sensing region 518 or by an optical sensor in a diagnostic consumable module configured to measure one or more properties of a sample in the optical sensing region 576. In some implementations, the temperature of the fluid in the sensing region 518 and/or the fluid in the optical sensing region 576 could be maintained at approximately body temperature, e.g., at approximately 37 degrees Celsius.

After calibration, the diagnostic module could instruct a user to inject a blood sample into the sample fluid input port 506. At least a portion of the blood sample could flow through the channel 538, the via 526, the channel 540 and into the sample fluid storage reservoir 508. A vacuum pump in the diagnostic module could be coupled to the pump connection port 522 through the pump hole 628. When this vacuum pump is turned on, the vacuum pump could draw the calibration fluid from the sensing region 518 into the waste fluid reservoir 520. Further, the vacuum pump could draw the blood sample from the sample fluid reservoir 508 and/or the channel 540 (if the fluid reservoir 508 is not vented), through the via 528, the channel 544, the bubble trap 514, the channel 546, the via 534, the channel 548, and into the sensing region 518. The diagnostic module and sensors 620 could then perform measurements on the blood sample to determine the concentration of certain analytes in the blood sample, for example. In this embodiment, the pump connection port 523 functions as a sample fluid displacement element of the diagnostic consumable that enables an external stimulus (vacuum pressure) to be applied to the diagnostic consumable in order to pump the fluid sample (whole blood) through the channel 102 and into the chamber 110. As noted earlier, in other embodiments other types of fluid displacement elements may be used to pump the blood sample through the channel 102, such as a pumping port or a mechanically actuatable air bladder fluidly connected upstream of the channel 102 and configured for application of a positive pressure source to push the blood sample through the channel.

Optical assays can be performed on the blood sample in the optical sensing region 576 on the diagnostic consumable 600. For example, a vacuum pump in the diagnostic module that is coupled to the pump connection port 523 through the pump hole 630 could be used to apply vacuum pressure to the pump connection port 523 to draw a portion of the blood sample from the sample fluid reservoir 508 and/or the channel 540, through the channel 542, the via 112, the haemolysis channel 102, and into the optical sensing region 576 within the chamber 110. As the blood sample is flowed through the haemolysis channel 102, it dissolves, re-suspends, and reacts with the haemolytic reagent that was dried-down on the array of micro-projections to generate haemolysed blood, which is then dispensed into the chamber 110. A light source and detector in the diagnostic module could then perform optical measurements on the haemolysed blood sample in the optical sensing region 576. In some embodiments, CO-oximetry could be performed in the optical sensing region 576 to measure the concentrations of total hemoglobin (tHb), oxyhemoglobin (O2HB), carboxyhemoglobin (COHb), methemoglobin (MetHb), deoxyhemoglobin (HHb), oxygen saturation (SO2) and/or total bilirubin (tBili) in the blood sample, for example. This could complete the testing that is performed using the diagnostic consumable 600. The diagnostic consumable 600 could be a disposable diagnostic device that is disposed of after use. However, reusable devices are also contemplated. As noted earlier, the same or similar structure as that of the haemolysis stage 100 could instead be used to mix a whole blood sample with a coagulant for an ACT test by using a coagulant rather than a haemolytic reagent and measuring the clotting time of the resulting mix of whole blood and coagulant downstream of the channel 102.

The embodiments described above relate primarily to diagnostic consumables. Other embodiments, including methods, are also contemplated.

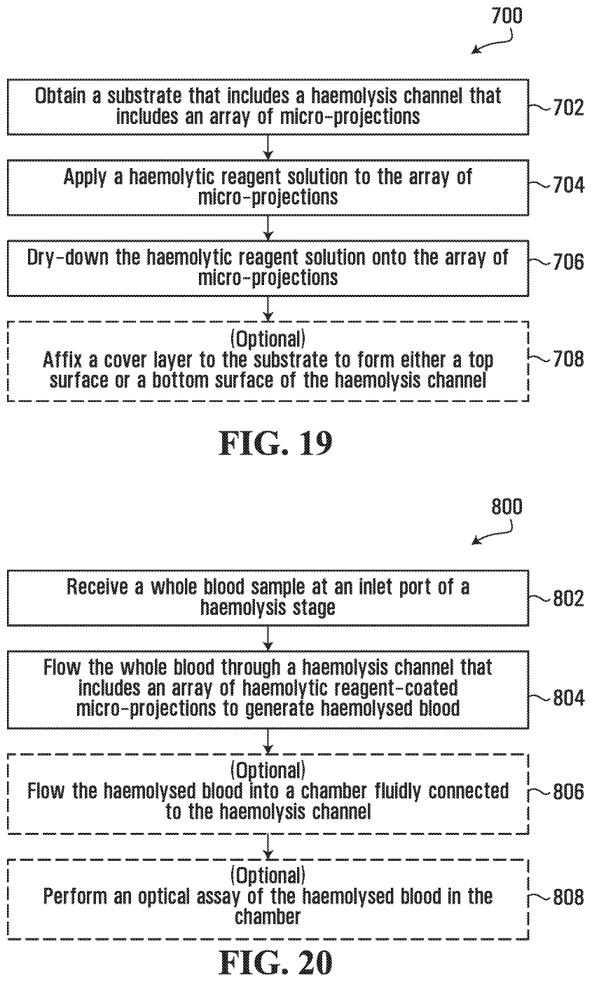

FIG. 19, for example, is a flow diagram illustrating an example method 700 for making a diagnostic consumable for use in analysis of a whole blood sample. In some implementations, the diagnostic consumable could be a microfluidic device. The method 700 includes multiple steps 702, 704, 706, 708.

Step 702 includes obtaining a substrate that includes a haemolysis channel having an array of micro-projections extending into the haemolysis channel to define a plurality of flow paths therebetween along at least a portion of a length of the haemolysis channel. In some embodiments, obtaining the substrate includes forming the substrate via a molding process, wherein the array of micro-projections is molded into the haemolysis channel in the molding process.

Step 704 includes applying a haemolytic reagent solution to the array of micro-projections in the haemolysis channel. Applying the haemolytic reagent solution could involve dispensing a predefined number of drops of the haemolytic reagent solution onto the array of micro-projections, for example. In some embodiments, capillarity of the array of micro-projections causes the haemolytic reagent solution to disperse amongst the array of micro-projections.

Step 706 includes drying-down the haemolytic reagent solution onto the array of micro-projections so that the array of micro-projections has dried haemolytic reagent disposed thereon. In some embodiments, the haemolytic reagent solution may be dried-down by allowing a solvent component of the haemolytic reagent solution to passively evaporate.

Step 708 is an optional step that includes affixing a cover layer to one side of the substrate to form either a top surface or a bottom surface of the haemolysis channel. The micro-projections extend into the haemolysis channel from the other of the top surface or the bottom surface of the haemolysis channel.

The example operations of the method 700 are illustrative of an example embodiment. Various ways to perform the illustrated operations, as well as examples of other operations that may be performed, are described herein. Further variations may be or become apparent.

For example, while the method 700 is illustrative of an example for making a diagnostic consumable that includes a haemolysis channel for preparing a haemolysed blood sample, similar operations may be performed to make other types of diagnostic consumables that include sample preparation channels for mixing and/or interacting a material with a fluid sample. For example, rather than applying haemolytic reagent solution to the array of micro-projections at step 704, a fluid comprising another material, such as a coagulant, may be applied to the micro-projections and the fluid may be dried-down onto the micro-projections at step 706 so that the array of micro-projections has the material disposed thereon.

FIG. 20 is a flow diagram illustrating an example method 800 for analysis of a whole blood sample on a diagnostic consumable. In some implementations, the diagnostic consumable could be a microfluidic device. The method 800 includes multiple steps 802, 804, 806, 808.

Step 802 includes receiving a whole blood sample at an inlet port of a haemolysis stage of the diagnostic consumable.

Step 804 includes haemolysing the whole blood by flowing the whole blood through a haemolysis channel of the haemolysis stage of the diagnostic consumable. The haemolysis channel includes an array of haemolytic reagent-coated micro-projections that extend into the haemolysis channel to define a plurality of flow paths therebetween. The haemolytic reagent interacts with the whole blood to generate haemolysed blood as the blood is flowed through the haemolysis channel. The haemolysis channel could be similar to the haemolysis channels 102, 202, 302 and/or 402 that are discussed in detail above, for example. In some embodiments, flowing the whole blood through the haemolysis channel includes pumping the whole blood through the haemolysis channel. For example, the whole blood could be pumped through the haemolysis channel by applying an external pressure source to the diagnostic consumable. The external pressure source could be a vacuum source that applies a vacuum to a vacuum port on the diagnostic card that is fluidly connected downstream of the haemolysis channel.

Step 806 is an optional step that includes flowing the haemolysed blood into a chamber that is fluidly connected to the haemolysis channel. The chamber could be similar to the chamber 110 discussed in detail above. For example, the chamber could be configured as a cuvette for use in an optical assay.

Step 808 is an optional step that includes performing an optical assay of the haemolysed blood in the chamber through at least a portion of the chamber that is optically transparent. For example, the chamber could have optically transparent top and bottom surfaces and performing the optical assay could involve performing a spectroscopic analysis of light passed through the haemolysed blood in the chamber via the optically transparent top and bottom surfaces of the chamber. Such an optical assay could be a CO-oximetry assay to measure the concentrations of tHb, O2HB, COHb, MetHb HHb and/or tBili in the blood sample, for example.

The example operations of the method 800 are illustrative of an example embodiment. Various ways to perform the illustrated operations, as well as examples of other operations that may be performed, are described herein. Further variations may be or become apparent.

For example, while the method 800 is illustrative of an example for analysis of a whole blood sample on a diagnostic consumable that includes a step of haemolysing the whole blood sample, similar operations may be performed for other types of analyses of whole blood samples or other types of fluid samples that require the mixing and/or interacting of one or more materials with the fluid sample. For example, similar to the operation at step 802, a fluid sample may be received at an inlet port of a sample preparation stage of a diagnostic consumable and then, similar to the operation at step 804, a material may be mixed into the fluid sample by flowing the fluid sample through a channel of the sample preparation stage that includes an array of micro-projections that have the material disposed thereon. As the fluid sample is flowed through the channel the material disposed on the micro-projections mixes with the fluid sample to generate a prepared fluid sample. For example, as described earlier, in some embodiments the fluid sample may be whole blood and the material disposed on the micro-projections may be a coagulant for mixing with the whole blood in order to perform a dotting time test.

Although the present disclosure relates primarily to haemolysis channels in diagnostic consumables for blood analysis systems, the embodiments described herein could also or instead relate to other types of fluid sample preparation channels in diagnostic consumables or other types of analysis systems. In particular, channels that include reagent-coated micro-projections could be used in any of a variety of applications where sample fluid preparation via reagent interaction on a diagnostic consumable would be advantageous. For example, an antibody reagent having an affinity for an antigen could be dried down on an array of micro-projections in a channel, so that when a sample fluid that may contain the antigen is flowed through the channel, any antigen that may be in the sample fluid is exposed to interact with the antibody reagent on the micro-projection array. Downstream analysis of the fluid dispensed from the channel may be performed to detect the antigen-antibody binding, for example.

### Illustrative Embodiments

The following provides a non-limiting list of additional Illustrative Embodiments of the present disclosure:

Example Embodiment 1. A diagnostic consumable for use in the analysis of a fluid sample, the diagnostic consumable comprising:

a substrate having a sample preparation stage, the sample preparation stage comprising:

i) an inlet port for receiving a fluid sample;

ii) an outlet port for dispensing a prepared fluid sample; and

iii) a channel extending from the inlet port to the outlet port, the channel comprising an array of micro-projections extending into the channel to define a plurality of flow paths therebetween along at least a portion of a length of the channel between the inlet port and the outlet port, the array of micro-projections having disposed thereon a material for mixing with the fluid sample as the fluid sample is flowed through the channel to generate the prepared fluid sample.

Example Embodiment 2. The diagnostic consumable of Example Embodiment 1, wherein the micro-projections of the array are arranged with a generally uniform spacing.
Example Embodiment 3. The diagnostic consumable of Example Embodiment 1 or 2, wherein the micro-projections of the array are disposed in staggered rows along at least a portion of the length of the channel, each row being arranged substantially transverse to a direction of flow through the channel.
Example Embodiment 4. The diagnostic consumable of Example Embodiment 3, wherein the staggered rows of micro-projections are disposed over substantially the entire length of the channel between the inlet port and the outlet port.
Example Embodiment 5. The diagnostic consumable of Example Embodiment 3, wherein the staggered rows of micro-projections comprises a first row of micro-projections and a second row of micro-projections disposed adjacently downstream from the first row of micro-projections relative to the direction of flow through the channel, the second row of micro-projections being offset in a direction transverse to the direction of flow through the haemolysis channel, relative to the first row of micro-projections, such that micro-projections in the second row are disposed substantially midway between micro-projections in the first row.
Example Embodiment 6. The diagnostic consumable of Example Embodiment 5, wherein:

a separation distance, measured transverse to the direction of flow through the haemolysis channel, between adjacent micro-projections in each of the first and second rows is substantially equal; and

the micro-projections in the first and second rows have a cross-sectional dimension, measured transverse to the direction of flow through the channel, that is greater than or equal to the separation distance between adjacent micro-projections in each of the first and second rows.

Example Embodiment 7. The diagnostic consumable of Example Embodiment 6, wherein:

the staggered rows of micro-projections further comprises a third row of micro-projections disposed adjacently downstream from the second row of micro-projections; and

micro-projections in the third row are substantially aligned, in the direction of flow through the channel, with micro-projections in the first row.

Example Embodiment 8. The diagnostic consumable of any of Example Embodiments 1 to 7, wherein:

the channel has a bottom surface, a top surface generally opposed to the bottom surface, and generally opposed side surfaces extending between the bottom surface and the top surface;

a height of the channel being defined as a distance between the bottom surface of the channel and the top surface of the channel; and

the micro-projections extend into the channel at least a portion of the height of the channel between the bottom surface and the top surface of the channel.

Example Embodiment 9. The diagnostic consumable of Example Embodiment 8, wherein the micro-projections extend the height of the channel between the bottom surface and the top surface of the channel.
Example Embodiment 10. The diagnostic consumable of Example Embodiment 9, wherein:

either the top surface or the bottom surface of the channel is formed by a cover layer affixed to one side of the substrate; and

the micro-projections extend from the other of the top surface and the bottom surface of the channel to the cover layer.

Example Embodiment 11. The diagnostic consumable of any one of Example Embodiments 1 to 10, further comprising a fluid displacement element in fluid communication with the channel, the fluid displacement element enabling an external stimulus to be applied to the diagnostic consumable to pump the fluid sample through the channel.
Example Embodiment 12. The diagnostic consumable of Example Embodiment 11, wherein the fluid displacement element comprises a vacuum port downstream of the channel, the vacuum port configured for application of a vacuum source to pump the fluid sample through the channel.
Example Embodiment 13. The diagnostic consumable of any one of Example Embodiments 1 to 12, wherein the material disposed on the array of micro-projections comprises a reagent that reacts with the fluid sample as the fluid sample is flowed through the channel.
Example Embodiment 14. The diagnostic consumable of Example Embodiment 13, wherein:

the fluid sample is whole blood;

the reagent disposed on the array of micro-projections comprises a haemolytic reagent; and

the prepared fluid sample comprises haemolysed blood.

Example Embodiment 15. The diagnostic consumable of Example Embodiment 13, wherein:

the fluid sample is whole blood;

the reagent disposed on the array of micro-projections comprises a coagulant; and

the prepared fluid sample comprises a mixture of the whole blood and the coagulant.

Example Embodiment 16. The diagnostic consumable of any one of Example Embodiments 1 to 15, wherein the substrate comprises a molded plastic substrate.
Example Embodiment 17. The diagnostic consumable of any one of Example Embodiments 1 to 16, wherein the micro-projections comprise micro-pillars.
Example Embodiment 18. The diagnostic consumable of any one of Example Embodiments 1 to 17, wherein the substrate further comprises a prepared fluid sample collection vessel, the prepared fluid sample collection vessel comprising:

an inlet port fluidly connected to the outlet port of the sample preparation stage for receiving the prepared fluid sample; and

a chamber for containing the prepared fluid sample.

Example Embodiment 19. A method for analysis of a fluid sample on a diagnostic consumable, the method comprising:

receiving a fluid sample at an inlet port of a sample preparation stage of the diagnostic consumable;

mixing a material into the fluid sample by flowing the fluid sample through a channel of the sample preparation stage of the diagnostic consumable, the channel comprising an array of micro-projections extending into the channel to define a plurality of flow paths therebetween along at least a portion of a length of the channel, the array of micro-projections having disposed thereon the material for mixing with the fluid sample as the fluid sample is flowed through the channel to generate a prepared fluid sample.

Example Embodiment 20. The method of Example Embodiment 19, wherein the method further comprises flowing the prepared fluid sample into a chamber on the diagnostic consumable that is fluidly connected to the channel.
Example Embodiment 21. The method of Example Embodiment 19 or 20, wherein flowing the fluid sample through the channel comprises applying an external stimulus to a fluid displacement element in fluid communication with the channel to pump the fluid sample through the channel.
Example Embodiment 22. The method of Example Embodiment 21, wherein the fluid displacement element comprises a pumping port in fluid communication with the channel, the pumping port being configured for application of an external pressure source to the diagnostic consumable to pump the fluid sample through the channel.
Example Embodiment 23. The method of Example Embodiment 22, wherein the pumping port comprises a vacuum port downstream of the channel, and wherein applying an external pressure source to diagnostic consumable comprises applying a vacuum source to the vacuum port to pump the fluid sample through the channel.
Example Embodiment 24. The method of any one of Example Embodiments 19 to 23, wherein the material disposed on the array of micro-projections comprises a reagent that reacts with the fluid sample as the fluid sample is flowed through the channel.
Example Embodiment 25. The method of Example Embodiment 24, wherein the reagent disposed on the array of micro-projections comprises a haemolytic reagent or a coagulant.
Example Embodiment 26. A method of making a diagnostic consumable for use in analysis of a fluid sample, the method comprising:

obtaining a substrate that includes a channel having an array of micro-projections extending into the channel to define a plurality of flow paths therebetween along at least a portion of a length of the channel;

applying a fluid to the array of micro-projections in the channel, the fluid comprising a material for deposition on the array of micro-projections; and

drying-down the fluid onto the array of micro-projections so that the array of micro-projections has the material disposed thereon.

Example Embodiment 27. The method of Example Embodiment 26, wherein applying the fluid to the array of micro-projections comprises dispensing a predefined number of drops of the fluid onto the array of micro-projections.
Example Embodiment 28. The method of Example Embodiment 26 or 27, wherein capillarity of the array of micro-projections causes the fluid to disperse amongst the array of micro-projections.
Example Embodiment 29. The method of any of Example Embodiments 26 to 28, wherein drying-down the fluid comprises passively evaporating a solvent component of the fluid.
Example Embodiment 30. The method of any of Example Embodiments 26 to 29, further comprising affixing a cover layer to one side of the substrate, the cover layer forming either a top surface or a bottom surface of the channel, the micro-projections extending into the channel from the other of the top surface or the bottom surface of the channel.
Example Embodiment 31. The method of any of Example Embodiments 26 to 30, wherein the material disposed on the array of micro-projections comprises a reagent that reacts with the fluid sample as the fluid sample is flowed through the channel.
Example Embodiment 32. The method of Example Embodiment 31, wherein the reagent disposed on the array of micro-projections comprises a haemolytic reagent or a coagulant.
Example Embodiment 33. The method of any one of Example Embodiments 26 to 32, wherein obtaining the substrate comprises forming the substrate via a molding process, the array of micro-projections being molded into the channel in the molding process.
Example Embodiment 34. The method of Example Embodiment 33, wherein the substrate comprises a plastic substrate and the molding process comprises injection molding.
Example Embodiment 35. The method of Example Embodiment 33 or 34, wherein forming the substrate via a molding process comprises molding the substrate such that the substrate comprises: an inlet port in fluid communication with the channel for receiving a fluid sample into the channel; and a pumping port in fluid communication with the channel for applying an external pressure source to the diagnostic consumable to pump the fluid sample through the channel.
Example Embodiment 36. The method of Example Embodiment 35, wherein the pumping port comprises a vacuum port formed in the substrate downstream of the channel, so that, in use, a vacuum source applied to the vacuum port causes the fluid sample to be pumped through the channel.
Example Embodiment 37. A diagnostic consumable for use in the analysis of whole blood, the diagnostic consumable comprising:

a substrate having a haemolysis stage, the haemolysis stage comprising:

i) an inlet port for receiving whole blood;

ii) an outlet port for dispensing haemolysed blood; and

iii) a haemolysis channel extending from the inlet port to the outlet port, the haemolysis channel comprising an array of micro-projections extending into the haemolysis channel to define a plurality of flow paths therebetween along at least a portion of a length of the haemolysis channel between the inlet port and the outlet port, the array of micro-projections having disposed thereon a haemolytic reagent for interaction with the whole blood as the whole blood is flowed through the haemolysis channel to generate haemolysed blood.

Example Embodiment 38. The diagnostic consumable of Example Embodiment 37, wherein the micro-projections of the array are arranged with a generally uniform spacing.
Example Embodiment 39. The diagnostic consumable of Example Embodiment 37 or 38, wherein the micro-projections of the array are disposed in staggered rows along at least a portion of the length of the haemolysis channel, each row being arranged substantially transverse to a direction of flow through the haemolysis channel.
Example Embodiment 40. The diagnostic consumable of Example Embodiment 39, wherein the staggered rows of micro-projections are disposed over substantially the entire length of the haemolysis channel between the inlet port and the outlet port.
Example Embodiment 41. The diagnostic consumable of Example Embodiment 39 or 40, wherein the micro-projections are disposed in the haemolysis channel such that:

in each row, a separation distance between adjacent micro-projections in the row is substantially equal; and

a separation distance between adjacent rows of micro-projections is substantially equal to the separation distance between adjacent micro-projections in each row.

Example Embodiment 42. The diagnostic consumable of any one of Example Embodiments 39 to 41, wherein the staggered rows of micro-projections comprises a first row of micro-projections and a second row of micro-projections disposed adjacently downstream from the first row of micro-projections relative to the direction of flow through the haemolysis channel, the second row of micro-projections being offset in a direction transverse to the direction of flow through the haemolysis channel, relative to the first row of micro-projections, such that micro-projections in the second row are disposed substantially midway between micro-projections in the first row.
Example Embodiment 43. The diagnostic consumable of Example Embodiment 42, wherein:

a separation distance, measured transverse to the direction of flow through the haemolysis channel, between adjacent micro-projections in each of the first and second rows is substantially equal; and

the micro-projections in the first and second rows have a cross-sectional dimension, measured transverse to the direction of flow through the haemolysis channel, that is greater than or equal to the separation distance between adjacent micro-projections in each of the first and second rows.

Example Embodiment 44. The diagnostic consumable of Example Embodiment 43, wherein:

the staggered rows of micro-projections further comprises a third row of micro-projections disposed adjacently downstream from the second row of micro-projections; and

micro-projections in the third row are substantially aligned, in the direction of flow through the haemolysis channel, with micro-projections in the first row.

Example Embodiment 45. The diagnostic consumable of any one of Example Embodiments 37 to 44, wherein:

the haemolysis channel has a bottom surface, a top surface generally opposed to the bottom surface, and generally opposed side surfaces extending between the bottom surface and the top surface;

a height of the haemolysis channel being defined as a distance between the bottom surface of the haemolysis channel and the top surface of the haemolysis channel; and

the micro-projections extend into the channel at least a portion of the height of the haemolysis channel between the bottom surface and the top surface of the haemolysis channel.

Example Embodiment 46. The diagnostic consumable of Example Embodiment 45, wherein the micro-projections extend the height of the haemolysis channel between the bottom surface and the top surface of the haemolysis channel.
Example Embodiment 47. The diagnostic consumable of Example Embodiment 46, wherein:

either the top surface or the bottom surface of the haemolysis channel is formed by a cover layer affixed to one side of the substrate; and

the micro-projections extend from the other of the top surface and the bottom surface of the haemolysis channel to the cover layer.

Example Embodiment 48. The diagnostic consumable of any one of Example Embodiments 37 to 47, wherein the substrate comprises a molded plastic substrate.
Example Embodiment 49. The diagnostic consumable of any one of Example Embodiments 37 to 48, wherein the micro-projections comprise micro-pillars.
Example Embodiment 50. The diagnostic consumable of Example Embodiment 49, wherein the micro-pillars have a generally circular cross-section.
Example Embodiment 51. The diagnostic consumable of any one of Example Embodiments 37 to 50, wherein the substrate further comprises a haemolysed blood collection vessel, the haemolysed blood collection vessel comprising:

an inlet port fluidly connected to the outlet port of the haemolysis stage for receiving the haemolysed blood; and

a chamber for containing the haemolysed blood.

Example Embodiment 52. The diagnostic consumable of Example Embodiment 51, wherein at least a portion of the chamber is optically transparent to permit an optical assay of the haemolysed blood.
Example Embodiment 53. The diagnostic consumable of Example Embodiment 52, wherein the chamber comprises:

optically transparent top and bottom surfaces, one of the optically transparent top and bottom surfaces of the chamber being formed by a cover layer affixed to one side of the substrate, the cover layer having an optically transparent window substantially aligned with the chamber.

Example Embodiment 54. The diagnostic consumable of Example Embodiment 53, wherein the other one of the optically transparent top and bottom surfaces of the chamber is molded into the substrate.
Example Embodiment 55. The diagnostic consumable of any one of Example Embodiments 51 to 54, wherein the substrate further comprises a vacuum port downstream of the haemolysed blood collection vessel, the vacuum port configured for application of a vacuum source to generate the flow of the whole blood through the haemolysis channel into the haemolysed blood collection vessel.
Example Embodiment 56. The diagnostic consumable of Example Embodiment 55, wherein the substrate further comprises a waste collection vessel for receiving excess haemolysed blood from the haemolysed blood collection vessel, the waste collection vessel being fluidly connected downstream of the haemolysed blood collection vessel and upstream of the vacuum port.
Example Embodiment 57. A method for analysis of a whole blood sample on a diagnostic consumable, the method comprising:

receiving a whole blood sample at an inlet port of a haemolysis stage of the diagnostic consumable;

haemolysing the whole blood by flowing the whole blood through a haemolysis channel of the haemolysis stage of the diagnostic consumable, the haemolysis channel comprising an array of micro-projections extending into the haemolysis channel to define a plurality of flow paths therebetween along at least a portion of a length of the haemolysis channel, the array of micro-projections having disposed thereon a haemolytic reagent for interaction with the whole blood as the whole blood is flowed through the haemolysis channel to generate haemolysed blood.

Example Embodiment 58. The method of Example Embodiment 57, wherein the method further comprises flowing the haemolysed blood into a chamber on the diagnostic consumable that is fluidly connected to the haemolysis channel.
Example Embodiment 59. The method of Example Embodiment 58, further comprising performing an optical assay of the haemolysed blood in the chamber through at least a portion of the chamber that is optically transparent.
Example Embodiment 60. The method of Example Embodiment 59, wherein the chamber comprises optically transparent top and bottom surfaces and performing the optical assay comprises performing a spectroscopic analysis of light passed through the haemolysed blood in the chamber via the optically transparent top and bottom surfaces of the chamber.
Example Embodiment 61. The method of any one of Example Embodiments 57 to 60, wherein flowing the whole blood through the haemolysis channel comprises pumping the whole blood through the haemolysis channel.
Example Embodiment 62. The method of Example Embodiment 61, wherein pumping the whole blood through the haemolysis channel comprises applying an external pressure source to the diagnostic consumable.
Example Embodiment 63. The method of any one of Example Embodiments 57 to 62, wherein applying an external pressure source to the diagnostic consumable comprises applying a vacuum source to a vacuum port on the diagnostic consumable that is fluidly connected to the haemolysis channel downstream of the haemolysis channel.
Example Embodiment 64. A method of making a diagnostic consumable for use in analysis of a whole blood sample, the method comprising:

obtaining a substrate that includes a haemolysis channel having an array of micro-projections extending into the haemolysis channel to define a plurality of flow paths therebetween along at least a portion of a length of the haemolysis channel;

applying a haemolytic reagent solution to the array of micro-projections in the haemolysis channel; and

drying-down the haemolytic reagent solution onto the array of micro-projections so that the array of micro-projections has dried haemolytic reagent disposed thereon.

Example Embodiment 65. The method of Example Embodiment 64, wherein applying the haemolytic reagent solution to the array of micro-projections comprises dispensing a predefined number of drops of the haemolytic reagent solution onto the array of micro-projections.
Example Embodiment 66. The method of Example Embodiment 64 or 65, wherein capillarity of the array of micro-projections causes the haemolytic reagent solution to disperse amongst the array of micro-projections.
Example Embodiment 67. The method of any one of Example Embodiments 64 to 66, wherein drying-down the haemolytic reagent solution comprises passively evaporating a solvent component of the haemolytic reagent solution.
Example Embodiment 68. The method of any one of Example Embodiments 64 to 67, further comprising affixing a cover layer to one side of the substrate, the cover layer forming either a top surface or a bottom surface of the haemolysis channel, the micro-projections extending into the haemolysis channel from the other of the top surface or the bottom surface of the haemolysis channel.
Example Embodiment 69. The method of any one of Example Embodiments 64 to 68, wherein obtaining the substrate comprises forming the substrate via a molding process, the array of micro-projections being molded into the haemolysis channel in the molding process.
Example Embodiment 70. The method of Example Embodiment 69, wherein the substrate comprises a plastic substrate and the molding process comprises injection molding.
Example Embodiment 71. A diagnostic consumable for use in the analysis of a fluid sample, the diagnostic consumable comprising:

a substrate having a sample preparation stage, the sample preparation stage comprising:

i) an inlet port for receiving a fluid sample;

ii) an outlet port for dispensing a prepared fluid sample; and

iii) a channel extending from the inlet port to the outlet port, the channel comprising a plurality of micro-projections extending into the channel to define a plurality of flow paths therebetween along at least a portion of a length of the channel between the inlet port and the outlet port, the plurality of micro-projections having disposed thereon a reagent for interaction with the fluid sample as the fluid sample is flowed through the channel to generate the prepared fluid sample.

Example Embodiment 72. The diagnostic consumable of Example Embodiment 71, wherein the micro-projections are spaced in a generally uniform array.
Example Embodiment 73. The diagnostic consumable of Example Embodiment 71 or 72, wherein the micro-projections are disposed in staggered rows along at least a portion of the length of the channel between the inlet port and the outlet port.
Example Embodiment 74. The diagnostic consumable of Example Embodiment 73, wherein the staggered rows of micro-projections are disposed over substantially the entire length of the channel between the inlet port and the outlet port.
Example Embodiment 75. The diagnostic consumable of Example Embodiment 73 or 74, wherein the micro-projections are disposed in the channel such that:

in each row, a separation distance between adjacent micro-projections in the row is substantially equal; and

a separation distance between adjacent rows of micro-projections is substantially equal to the separation distance between adjacent micro-projections in each row.

Example Embodiment 76. The diagnostic consumable of Example Embodiment 37 or Example Embodiment 74, wherein the staggered rows of micro-projections comprises a first row of micro-projections and a second row of micro-projections disposed adjacently downstream from the first row of micro-projections relative to the direction of flow through the channel, the second row of micro-projections being offset in a direction transverse to the direction of flow through the channel, relative to the first row of micro-projections, such that micro-projections in the second row are disposed substantially midway between micro-projections in the first row.
Example Embodiment 77. The diagnostic consumable of Example Embodiment 76, wherein:

a separation distance, measured transverse to the direction of flow through the channel, between adjacent micro-projections in each of the first and second rows is substantially equal; and

the micro-projections in the first and second rows have a cross-sectional dimension, measured transverse to the direction of flow through the channel, that is greater than or equal to the separation distance between adjacent micro-projections in each of the first and second rows.

Example Embodiment 78. The diagnostic consumable of Example Embodiment 77, wherein:

the staggered rows of micro-projections further comprises a third row of micro-projections disposed adjacently downstream from the second row of micro-projections; and

micro-projections in the third row are substantially aligned, in the direction of flow through the channel, with micro-projections in the first row.

Example Embodiment 79. The diagnostic consumable of any one of Example Embodiments 76 to 78, wherein:

the haemolysis channel has a bottom surface, a top surface generally opposed to the bottom surface, and generally opposed side surfaces extending between the bottom surface and the top surface;

a height of the channel being defined as a distance between the bottom surface of the channel and the top surface of the channel; and

the micro-projections extend into the channel at least a portion of the height of the channel between the bottom surface and the top surface of the channel.

Example Embodiment 80. The diagnostic consumable of Example Embodiment 79, wherein the micro-projections extend the height of the channel between the bottom surface and the top surface of the channel.
Example Embodiment 81. The diagnostic consumable of Example Embodiment 80, wherein:

either the top surface or the bottom surface of the channel is formed by a cover layer affixed to one side of the substrate; and

the micro-projections extend from the other of the top surface and the bottom surface of the channel to the cover layer.

Example Embodiment 82. The diagnostic consumable of any one of Example Embodiments 71 to 81, wherein the substrate comprises a molded plastic substrate.
Example Embodiment 83. The diagnostic consumable of any one of Example Embodiments 71 to 82, wherein the micro-projections comprise micro-pillars.
Example Embodiment 84. The diagnostic consumable of Example Embodiment 83, wherein the micro-pillars have a generally circular cross-section.
Example Embodiment 85. The diagnostic consumable of any one of Example Embodiments 71 to 84, wherein the substrate further comprises a prepared sample collection vessel, the prepared sample collection vessel comprising:

an inlet port fluidically connected the outlet port of the sample preparation stage for receiving the prepared fluid sample; and

a chamber for containing the prepared fluid sample.

Example Embodiment 86. The diagnostic consumable of Example Embodiment 85, wherein at least a portion of the chamber is optically transparent to permit an optical assay of the prepared fluid sample.
Example Embodiment 87. The diagnostic consumable of Example Embodiment 86, wherein the chamber comprises:

optically transparent top and bottom surfaces, one of the optically transparent top and bottom surfaces of the chamber being formed by a cover layer affixed to one side of the substrate, the cover layer having an optically transparent window substantially aligned with the chamber.

Example Embodiment 88. The diagnostic consumable of Example Embodiment 87, wherein the other one of the optically transparent top and bottom surfaces of the chamber is molded into the substrate.
Example Embodiment 89. The diagnostic consumable of any one of Example Embodiments 85 to 88, wherein the substrate further comprises a vacuum port downstream of the prepared sample collection vessel, the vacuum port configured for application of a vacuum source to generate the flow of the fluid sample through the channel of the sample preparation stage into the prepared sample collection vessel.
Example Embodiment 90. The diagnostic consumable of Example Embodiment 89, wherein the substrate further comprises a waste collection vessel for receiving excess prepared fluid sample from the prepared fluid sample collection vessel, the waste collection vessel being fluidically connected downstream of the prepared fluid sample collection vessel and upstream of the vacuum port.
Example Embodiment 91. The diagnostic consumable of any one of Example Embodiments 71 to 90, wherein the sample preparation stage comprises a lysis stage and the reagent comprises a lysing reagent.
Example Embodiment 92. The diagnostic consumable of Example Embodiment 91, wherein:

the fluid sample is whole blood;

the reagent comprises a haemolytic reagent; and

the prepared fluid sample comprises haemolysed blood.

Example Embodiment 93. The diagnostic consumable of any one of Example Embodiments 71 to 90, wherein the reagent comprises at least one antibody.

The inventive concepts disclosed herein are not limited in their application to the details of construction and the arrangement of the components set forth in the description or illustrated in the drawings. The inventive concepts disclosed herein are capable of other embodiments or of being practiced or carried out in various ways. Also, it is to be understood that the phraseology and terminology employed herein is for the purpose of description and should not be regarded as limiting the inventive concepts disclosed and claimed herein in any way.

Numerous specific details are set forth in order to provide a more thorough understanding of the inventive concepts. However, it will be apparent to one of ordinary skill in the art that the inventive concepts within the instant disclosure may be practiced without these specific details. In other instances, well-known features have not been described in detail to avoid unnecessarily complicating the instant disclosure.

As used herein, the terms “comprises,” “comprising,” “includes,” “including,” “has,” “having” or any other variation thereof, are intended to cover a nonexclusive inclusion. For example, a composition, a process, method, article, or apparatus that comprises a list of elements is not necessarily limited to only those elements but may include other elements not expressly listed or inherently present therein.

As used herein the terms “approximately,” “about,” “substantially” and variations thereof are intended to include not only the exact value qualified by the term, but to also include some slight deviations therefrom, such as deviations caused by measuring error, manufacturing tolerances, wear and tear on components or structures, stress exerted on structures, and combinations thereof, for example.

Unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present). An inclusive or may be understood as being the equivalent to: at least one of condition A or B.

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

Any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

1. A diagnostic consumable for use in the analysis of a fluid sample, the diagnostic consumable comprising:
a substrate having a sample preparation stage, the sample preparation stage comprising:
i) an inlet port for receiving a fluid sample;
ii) an outlet port for dispensing a prepared fluid sample; and
iii) a channel extending from the inlet port to the outlet port, the channel comprising an array of micro-projections extending into the channel to define a plurality of flow paths therebetween along at least a portion of a length of the channel between the inlet port and the outlet port, the array of micro-projections having disposed thereon a material for mixing with the fluid sample as the fluid sample is flowed through the channel to generate the prepared fluid sample.

2. The diagnostic consumable of claim 1, wherein the micro-projections of the array are arranged with a generally uniform spacing.

3. The diagnostic consumable of claim 1, wherein the micro-projections of the array are disposed in staggered rows along at least a portion of the length of the channel, each row being arranged substantially transverse to a direction of flow through the channel.

4. The diagnostic consumable of claim 3, wherein the staggered rows of micro-projections are disposed over substantially the entire length of the channel between the inlet port and the outlet port.

5. The diagnostic consumable of claim 3, wherein the staggered rows of micro-projections comprises a first row of micro-projections and a second row of micro-projections disposed adjacently downstream from the first row of micro-projections relative to the direction of flow through the channel, the second row of micro-projections being offset in a direction transverse to the direction of flow through the haemolysis channel, relative to the first row of micro-projections, such that micro-projections in the second row are disposed substantially midway between micro-projections in the first row.

6. The diagnostic consumable of claim 5, wherein:
a separation distance, measured transverse to the direction of flow through the haemolysis channel, between adjacent micro-projections in each of the first and second rows is substantially equal; and
the micro-projections in the first and second rows have a cross-sectional dimension, measured transverse to the direction of flow through the channel, that is greater than or equal to the separation distance between adjacent micro-projections in each of the first and second rows.

7. The diagnostic consumable of claim 6, wherein:
the staggered rows of micro-projections further comprises a third row of micro-projections disposed adjacently downstream from the second row of micro-projections; and
micro-projections in the third row are substantially aligned, in the direction of flow through the channel, with micro-projections in the first row.

8. The diagnostic consumable of claim 1, wherein:
the channel has a bottom surface, a top surface generally opposed to the bottom surface, and generally opposed side surfaces extending between the bottom surface and the top surface;
a height of the channel being defined as a distance between the bottom surface of the channel and the top surface of the channel; and
the micro-projections extend into the channel at least a portion of the height of the channel between the bottom surface and the top surface of the channel.

9. The diagnostic consumable of claim 8, wherein the micro-projections extend the height of the channel between the bottom surface and the top surface of the channel.

10. The diagnostic consumable of claim 9, wherein:
either the top surface or the bottom surface of the channel is formed by a cover layer affixed to one side of the substrate; and
the micro-projections extend from the other of the top surface and the bottom surface of the channel to the cover layer.

11. The diagnostic consumable of claim 1, further comprising a fluid displacement element in fluid communication with the channel, the fluid displacement element enabling an external stimulus to be applied to the diagnostic consumable to pump the fluid sample through the channel.

12. The diagnostic consumable of claim 11, wherein the fluid displacement element comprises a vacuum port downstream of the channel, the vacuum port configured for application of a vacuum source to pump the fluid sample through the channel.

13. The diagnostic consumable of claim 1, wherein the material disposed on the array of micro-projections comprises a reagent that reacts with the fluid sample as the fluid sample is flowed through the channel.

14. The diagnostic consumable of claim 13, wherein:
the fluid sample is whole blood;
the reagent disposed on the array of micro-projections comprises a haemolytic reagent; and
the prepared fluid sample comprises haemolysed blood.

15. The diagnostic consumable of claim 13, wherein:
the fluid sample is whole blood;
the reagent disposed on the array of micro-projections comprises a coagulant; and
the prepared fluid sample comprises a mixture of the whole blood and the coagulant.

16. The diagnostic consumable of claim 1, wherein the substrate comprises a molded plastic substrate.

17. The diagnostic consumable of claim 1, wherein the micro-projections comprise micro-pillars.

18. The diagnostic consumable of claim 1, wherein the substrate further comprises a prepared fluid sample collection vessel, the prepared fluid sample collection vessel comprising:
an inlet port fluidly connected to the outlet port of the sample preparation stage for receiving the prepared fluid sample; and
a chamber for containing the prepared fluid sample.

19. A method for analysis of a fluid sample on a diagnostic consumable, the method comprising:
receiving a fluid sample at an inlet port of a sample preparation stage of the diagnostic consumable;
mixing a material into the fluid sample by flowing the fluid sample through a channel of the sample preparation stage of the diagnostic consumable, the channel comprising an array of micro-projections extending into the channel to define a plurality of flow paths therebetween along at least a portion of a length of the channel, the array of micro-projections having disposed thereon the material for mixing with the fluid sample as the fluid sample is flowed through the channel to generate a prepared fluid sample.

20. The method of claim 19, wherein the method further comprises flowing the prepared fluid sample into a chamber on the diagnostic consumable that is fluidly connected to the channel.

21. The method of claim 19, wherein flowing the fluid sample through the channel comprises applying an external stimulus to a fluid displacement element in fluid communication with the channel to pump the fluid sample through the channel.

22. The method of claim 21, wherein the fluid displacement element comprises a pumping port in fluid communication with the channel, the pumping port being configured for application of an external pressure source to the diagnostic consumable to pump the fluid sample through the channel.

23. The method of claim 22, wherein the pumping port comprises a vacuum port downstream of the channel, and wherein applying an external pressure source to diagnostic consumable comprises applying a vacuum source to the vacuum port to pump the fluid sample through the channel.

24. The method of claim 19, wherein the material disposed on the array of micro-projections comprises a reagent that reacts with the fluid sample as the fluid sample is flowed through the channel.

25. The method of claim 24, wherein the reagent disposed on the array of micro-projections comprises a haemolytic reagent or a coagulant.

26. A method of making a diagnostic consumable for use in analysis of a fluid sample, the method comprising:
obtaining a substrate that includes a channel having an array of micro-projections extending into the channel to define a plurality of flow paths therebetween along at least a portion of a length of the channel;
applying a fluid to the array of micro-projections in the channel, the fluid comprising a material for deposition on the array of micro-projections; and
drying-down the fluid onto the array of micro-projections so that the array of micro-projections has the material disposed thereon.

27. The method of claim 26, wherein applying the fluid to the array of micro-projections comprises dispensing a predefined number of drops of the fluid onto the array of micro-projections.

28. The method of claim 26, wherein capillarity of the array of micro-projections causes the fluid to disperse amongst the array of micro-projections.

29. The method of claim 26, wherein drying-down the fluid comprises passively evaporating a solvent component of the fluid.

30. The method of claim 26, further comprising affixing a cover layer to one side of the substrate, the cover layer forming either a top surface or a bottom surface of the channel, the micro-projections extending into the channel from the other of the top surface or the bottom surface of the channel.

31. The method of claim 26, wherein the material disposed on the array of micro-projections comprises a reagent that reacts with the fluid sample as the fluid sample is flowed through the channel.

32. The method of claim 31, wherein the reagent disposed on the array of micro-projections comprises a haemolytic reagent or a coagulant.

33. The method of claim 26, wherein obtaining the substrate comprises forming the substrate via a molding process, the array of micro-projections being molded into the channel in the molding process.

34. The method of claim 33, wherein the substrate comprises a plastic substrate and the molding process comprises injection molding.

35. The method of claim 33, wherein forming the substrate via a molding process comprises molding the substrate such that the substrate comprises: an inlet port in fluid communication with the channel for receiving a fluid sample into the channel; and a pumping port in fluid communication with the channel for applying an external pressure source to the diagnostic consumable to pump the fluid sample through the channel.

36. The method of claim 35, wherein the pumping port comprises a vacuum port formed in the substrate downstream of the channel, so that, in use, a vacuum source applied to the vacuum port causes the fluid sample to be pumped through the channel.
